(19) United States
(12) Patent Application Publication
(10) Pub. No.: US 2018/0117268 A1
(43) Pub. Date: May 3, 2018

(54) VAPORIZER, APPARATUS, DEVICE, AND METHODS

(71) Applicant: PODA TECHNOLOGIES LTD., Vancouver (CA)

(72) Inventors: Ryan Daniel SELBY, Vancouver (CA); Ryan KARKAIRAN, Vancouver (CA)

(73) Assignee: PODA TECHNOLOGIES LTD., Vancouver (CA)

(21) Appl. No.: 15/569,909

(22) PCT Filed: Apr. 29, 2016
(86) PCT No.: PCT/CA2016/050504

§ 371 Date: Oct. 27, 2017

Related U.S. Application Data
(60) Provisional application No. 62/154,692, filed on Apr. 29, 2015.

(51) Int. Cl. A61M 11/04; A61M 15/06; A24F 47/00

(52) U.S. Cl. CPC A61M 11/042; A61M 15/06; A61M 2205/127; A61M 2205/123; A24F 47/008

(57) ABSTRACT

A vaporizer cartridge includes a base, inner and outer substantially concentric walls extending from the base and defining a vaporization chamber between the outer and inner walls adapted for containing at least one vaporizable substance, and a heating receptacle opening extending through the base within a perimeter of the inner wall adapted for receiving a heating element inserted into the heating receptacle opening, wherein a top edge of the outer wall forms an open top rim adapted for coupling to a mouthpiece. The vaporizer cartridge may also have a mouthpiece with a top opening, a central open bore, and a bottom opening sealably coupled to the open top rim. A vaporizer apparatus having a heating component and a vaporizer cartridge is provided, as is a method of making a vaporizer cartridge and a method of vaporizing a consumable substance.

### CROSS REFERENCE TO RELATED APPLICATIONS

This application is related and claims priority to U.S. Provisional Patent Application Ser. No. 62/154,692 filed Apr. 29, 2015 and entitled “Vaporizer Apparatus, Device and Methods”, which is hereby incorporated herein by reference in its entirety for all purposes.

### TECHNICAL FIELD

The present disclosure relates generally to vaporizers. More particularly, the present disclosure relates to cartridges for vaporizers. Even more particularly, the present disclosure relates to cartridges for removable attachment to heating components of vaporizers.

### BACKGROUND

Vaporization is a process that involves converting liquids or solids to a gas or vapor without combustion. When a solid is converted to gas or vapor, that phenomenon is called sublimation. When a liquid is converted to gas or vapor, that phenomenon is called evaporation. Collectively, sublimation and evaporation are referred to as vaporizing, vaporization, and the like, interchangeably, throughout this disclosure.

In the related art, a vaporizer is a device used to vaporize active ingredients of a consumable substance for the purpose of inhalation. Consumable substances include medicated waxes, e-liquids (such as glycol based vaporizable liquids commonly used in “e-cigarette” or “vaping” apparatus), medicated oils, and herbs, including plant materials (typically dried plant materials), such as cannabis, tobacco, eucalyptus, shisha, blends of plant materials, and other vaporizable substances. Generally, a vaporizer heats the consumable substance until the active ingredients of the consumable substance are released, or vaporized, typically by reaching the boiling point of the consumable substance.

Related art vaporizers may be advantageous to conventional techniques for consuming substances, such as smoking or combustion. It is generally known that smoking substances causes damage to a user's respiratory system and may delivery carcinogens as well as other harmful substances associated with the combustion or partial combustion of a consumable substance into a user's body. Vaporizers which typically operate at temperatures below the combustion point of the consumable substance may desirably reduce the amount of smoke emitted and the amount of smoke consumed by the user.

Current related art vaporizers are traditionally large, stationary devices that are difficult to transport. For example, consider the Volcano® vaporizer apparatus that is produced by Storz and Bickel®. The Volcano® vaporizer has a relatively heavy base that requires an electrical outlet and vapor bag to collect the vapors. This configuration requires users to remain at home and near an electrical outlet to operate the related art vaporizer. Some portable vaporizers have also been developed, such as the “Portable Hand-Held Vaporizer Heating Assembly” disclosed in U.S. Pat. No. 8,739,786. These portable vaporizers generally utilize a convection-based heating method which is slow to heat and suffers from decreased efficiency after continued use. As such, a need exists for a vaporizer apparatus that addresses the foregoing problems of the related art.

### SUMMARY

The present disclosure involves a vaporizer cartridge and vaporizer apparatus that addresses some of the problems experienced in the related art. In accordance with an embodiment of the present disclosure, a vaporizer cartridge is provided. The vaporizer cartridge comprises a base, inner and outer substantially concentric walls extending from the base and defining a vaporization chamber between said outer and inner walls adapted for containing at least one vaporizable substance, and a heating receptacle opening extending through the base within a perimeter of the inner wall adapted for receiving a heating element inserted into the heating receptacle opening, where a top edge of the outer wall forms an open top rim adapted for coupling to a mouthpiece.

In a particular embodiment, the vaporizer cartridge additionally comprises a mouthpiece comprising a top opening, a central open bore, and a bottom opening sealably coupled to the open top rim of the cartridge. In another embodiment, the vaporizer cartridge additionally comprises at least one seal situated between the bottom opening of the mouthpiece and the open top rim of said cartridge. In a further embodiment, the mouthpiece is releasably coupled to the open top rim of the cartridge. In yet a further embodiment, the mouthpiece is lockably coupled to thed open top rim of the cartridge, such as by one or more mechanical locking members. In another embodiment, the base of the vaporizer cartridge additionally comprises at least one air aperture adapted to admit air into the vaporization chamber. And in a further embodiment, the vaporizer cartridge additionally comprises a guard layer surrounding at least a portion of the outer wall, where the guard layer is adapted to at least partially thermally insulate a user grasping the guard layer from the outer wall. In another embodiment, the vaporizer cartridge additionally comprises at least one electronically readable memory device attached to the cartridge, such as at least one of: a printed addressable memory device, a thin film printed memory device, a radio frequency identification device, a barcode and a magnetic memory device. In a further embodiment, the vaporizer cartridge additionally comprises at least one vaporizable material contained in the vaporization chamber, such as a consumable substance comprising at least one of: herbal, tobacco, cannabis, and glycol-based liquid vaporizable materials.

In accordance with another embodiment of the present disclosure, a vaporizer apparatus is provided. In one such embodiment, the vaporizer apparatus comprises a vaporizer cartridge comprising a base, inner and outer substantially concentric walls extending from the base and defining a vaporization chamber between said outer and inner walls adapted for containing at least one vaporizable substance, and a heating receptacle opening extending through the base within a perimeter of the inner wall adapted for receiving a heating element inserted into the heating receptacle opening, where a top edge of the outer wall forms an open top rim adapted for coupling to a mouthpiece. The vaporizer apparatus also comprises a heating component comprising a heating element extending from a top surface thereof and circuitry adapted to control heating of the heating element, where the heating element is inserted into the heating receptacle opening in the base of the vaporizer cartridge, and the vaporizer cartridge is coupled to the top surface of the heating component.

In another embodiment, the vaporizer cartridge of the vaporizer apparatus additionally comprises a mouthpiece comprising a top opening, a central open bore, and a bottom opening, sealably coupled to the open top rim of the cartridge. In a further embodiment, the vaporizer apparatus circuitry comprises a battery and a power switch adapted for activation by a user, and/or the heating component is adapted for handheld use by a user. In yet a further embodiment, the vaporizer cartridge additionally comprises at least one electronically readable memory device attached to the cartridge, and the heating component additionally comprises at least one electronic memory device reader adapted to interface with the electronically readable memory device attached to the cartridge.

In accordance with another embodiment of the present disclosure, a method of making a vaporizer cartridge is provided. In one such embodiment, the method comprises:

providing a base, inner and outer substantially concentric walls extending from the base and defining a vaporization chamber between the outer and inner walls adapted for containing at least one vaporizable substance, and a heating receptacle opening extending through the base within a perimeter of the inner wall adapted for receiving a heating element inserted into the heating receptacle opening, wherein a top edge of the outer wall forms an open top rim adapted for coupling to a mouthpiece;

filling at least a portion of said vaporization chamber with at least one vaporizable substance; and

sealably coupling a mouthpiece comprising a top opening, a central open bore, and a bottom opening to the open top rim of the cartridge.

In accordance with yet a further embodiment of the present disclosure, a method of vaporizing a consumable substance is provided. In one such embodiment, the method comprises:

providing a vaporizer cartridge comprising a base, inner and outer substantially concentric walls extending from the base and defining a vaporization chamber between the outer and inner walls and containing at least one vaporizable consumable substance, and a heating receptacle opening extending through the base within a perimeter of the inner wall adapted for receiving a heating element inserted into the heating receptacle opening, wherein a top edge of the outer wall forms an open top rim sealably coupled to a mouthpiece;

providing a heating component comprising a heating element extending from a top surface thereof and circuitry adapted to control heating of the heating element;

coupling the vaporizer cartridge to the heating component such that the heating element is inserted into the heating receptacle opening in the base of the vaporizer cartridge; and

activating the heating component to heat the heating element and vaporize the at least one consumable substance.

In yet further embodiments in accordance with the present disclosure, a vaporizer apparatus has a portable non-combustion vaporizer device for generating vapors, wherein the vaporizer device comprises: a heating component and a vaporizer cartridge. The heating component comprises a heating element substantially perpendicularly extending therefrom and into the vaporizer cartridge. In an embodiment of the present disclosure, the cartridge is removably attachable in relation to the heating component. In an embodiment of the present disclosure, the device further comprises a collar for facilitating removably attaching the cartridge to the heating component.

In another embodiment of the present disclosure, the cartridge is adapted to store and vaporize at least one consumable substance. The cartridge comprises a mouth piece and a storage chamber. The storage chamber comprises a dome, a tube, or a heating element receptacle, for receiving a heating element, such as a heating component. In some embodiments of the present disclosure, the storage chamber comprises an air permeable base. In some embodiments of the present disclosure, the storage chamber comprises a thermally conductive material for facilitating distributing heat throughout the at least one consumable substance. In some embodiments, the mouthpiece and the storage chamber are removably attachable in relation to one another.

In some embodiments of the present disclosure, the cartridge comprises at least one pre-filled consumable substance. In some embodiments of the present disclosure, the at least one consumable substance comprises tobacco. In some embodiments of the present disclosure, the at least one consumable substance comprises at least one of herbal materials, tobacco, cannabis materials and e-juice or glycol based liquid materials.

In another embodiment of the present disclosure, the mouth piece comprises a vapor-permeable base and a conically-shaped outer surface. In some embodiments of the present disclosure, the vapor-permeable base comprises a non-vapor-permeable material having one or more apertures. In some embodiments of the present disclosure, the mouth piece comprises a vapor-permeable liner.

In an embodiment of the present disclosure, a method of fabricating a portable non-combustion vaporizer device for generating vapors comprises providing a heating component and providing a cartridge. The heating component providing comprises providing a heating element substantially perpendicularly extending therefrom and into the cartridge. In an embodiment of the present disclosure, the cartridge providing comprises providing the cartridge as removably attachable in relation to the heating component. In an embodiment of the present disclosure, the method of fabricating the portable non-combustion vaporizer device further comprises providing a collar for facilitating removably attaching the cartridge to the heating component.

In an embodiment of the present disclosure, a method of generating vapors by way of a portable non-combustion vaporizer device comprises providing the vaporizer device, the device providing comprising providing a heating component and providing a cartridge. The heating component providing comprises providing a heating element substantially perpendicularly extending therefrom and into the cartridge. In an embodiment of the present disclosure, the cartridge providing comprises providing the cartridge as removably attachable in relation to the heating component. The cartridge providing comprises loading cartridge, wherein loading the cartridge comprises loading at least one consumable substance. In an embodiment of the present disclosure, the method of providing the portable non-combustion vaporizer device further comprises providing a collar for facilitating removably attaching the cartridge to the heating component. The method of generating vapors further comprises coupling the cartridge with the heating component by way of the collar. The method of generating vapors further comprises providing a mouthpiece and coupling the mouthpiece with the collar. The method of generating vapors further comprises powering the heating component, thereby applying heat to the at least one consumable substance disposed in the cartridge.

Advantages of the present disclosure will become apparent when considering the drawings in conjunction with the below Detailed Description.

### BRIEF DESCRIPTION OF THE DRAWING

The above, and other, aspects, features, and advantages of several embodiments of the present disclosure will be more apparent from the following Detailed Description as presented in conjunction with the following several figures of the Drawing.

The apparatus and methods of the present disclosure will now be described with reference to the several accompanying figures of the Drawing, as follows.

FIG. 1A is a diagram illustrating a front elevation perspective view of a vaporizer, in accordance with an embodiment of the present disclosure.

FIG. 1B is a diagram illustrating a cut-away front elevation perspective view of a vaporizer, in accordance with a further embodiment of the present disclosure.

FIG. 2A is a diagram illustrating a front elevation perspective view of a heating component, in accordance with an embodiment of the present disclosure.

FIG. 2B is a diagram illustrating a front elevation perspective view of the heating component, as shown in FIG. 2A, with the outer surface shown as transparent for furthering understanding of any internal components, in accordance with an embodiment of the present disclosure.

FIG. 2C is a diagram illustrating a cut-away front elevation perspective view of a heating component for a vaporizer, as shown in FIG. 2A, in accordance with a further embodiment of the present disclosure.

FIG. 3 is a diagram illustrating a front elevation perspective view of a collar, in accordance with an embodiment of the present disclosure.

FIG. 4A is a diagram illustrating a front elevation perspective view of a cartridge, with the outer surface shown as transparent for furthering understanding of any internal components, in accordance with an embodiment of the present disclosure.

FIG. 4B is a diagram illustrating an exploded front elevation perspective view of the cartridge, as shown in FIG. 4A, with the outer surface shown as transparent for furthering understanding of any internal components, in accordance with an embodiment of the present disclosure.

FIG. 4C is a diagram illustrating a cut-away front elevation perspective view of a cartridge, in accordance with a further embodiment of the present disclosure.

FIG. 5 is a diagram illustrating a front elevation perspective view of a vaporizer device, in accordance with an embodiment of the present disclosure.

FIG. 6A is a diagram illustrating a front elevation perspective view of a circuitry component, in accordance with an embodiment of the present disclosure.

FIG. 6B is a diagram illustrating a front elevation perspective view of the circuitry component, as shown in FIG. 6A, with the outer surface shown as transparent for furthering understanding of any internal components, in accordance with an embodiment of the present disclosure.

FIG. 7 is a diagram illustrating a cut-away front elevation perspective view of a protective adapter, in accordance with an embodiment of the present disclosure.

FIG. 8 is a diagram illustrating a front elevation perspective view of an adapter and heating element, in accordance with an embodiment of the present disclosure.

FIG. 9A is a diagram illustrating a top view of an adapter, in accordance with an embodiment of the present disclosure.

FIG. 9B is a diagram illustrating a front elevation perspective view of the adapter, as shown in FIG. 9A, in accordance with an embodiment of the present disclosure.

FIG. 9C is a diagram illustrating a cut-away front elevation perspective view of the adapter, as shown in FIG. 9A, in accordance with an embodiment of the present disclosure.

FIG. 10 is a diagram illustrating a front view of a heating element, in accordance with an embodiment of the present disclosure.

FIG. 11A is a diagram illustrating a front elevation perspective view of a protective shell, in accordance with an embodiment of the present disclosure.

FIG. 11B is a diagram illustrating a cut-away front view of the protective shell, as shown in FIG. 11A, in accordance with an embodiment of the present disclosure.

FIG. 12A is a diagram illustrating a cut-away front elevation perspective view of a cartridge, in accordance with an embodiment of the present disclosure.

FIG. 12B is a diagram illustrating an exploded cut-away front elevation perspective view of the cartridge, as shown in FIG. 12A, in accordance with an embodiment of the present disclosure.

FIG. 13A, is a diagram illustrating a cut-away front elevation perspective view of a vaporization chamber, in accordance with an embodiment of the present disclosure.

FIG. 13B is a diagram illustrating a cut-away front elevation perspective view of the vaporization chamber, as shown in FIG. 13A, with the outer ring removed for furthering understanding of any internal components, in accordance with an embodiment of the present disclosure.

FIG. 13C is a diagram illustrating a cut-away front elevation perspective view of the vaporization chamber, as shown in FIG. 13A, with the outer ring and middle heating receptacle removed for furthering understanding of any internal components, in accordance with an embodiment of the present disclosure.

FIG. 13D is a diagram illustrating a cut-away front elevation perspective view of a cartridge including an internal vaporization chamber, in accordance with a further embodiment of the present disclosure.

FIG. 14 is a diagram illustrating a top perspective view of a protective shell, in accordance with an embodiment of the present disclosure.

FIG. 15A is a diagram illustrating a cut-away front elevation perspective view of a vaporization chamber, in accordance with an embodiment of the present disclosure.

FIG. 15B is a diagram illustrating a cut-away front elevation perspective view of the vaporization chamber, as shown in FIG. 15A, with the outer ring removed for furthering understanding of any internal components, in accordance with an embodiment of the present disclosure.

FIG. 15C is a diagram illustrating a cut-away front elevation perspective cross-section view of the vaporization chamber, as shown in FIG. 15A, with the outer ring and middle heating receptacle removed for furthering understanding of any internal components, in accordance with an embodiment of the present disclosure.

FIG. 16 is a diagram illustrating a top perspective view of a protective shell, in accordance with an embodiment of the present disclosure.

FIG. 17A is a diagram illustrating a cut-away front perspective view of a mouthpiece, in accordance with an embodiment of the present disclosure.

FIG. 17B is a diagram illustrating in a front perspective view of the mouthpiece, as shown in FIG. 17A, in accordance with an embodiment of the present disclosure.

FIG. 18 is a flowchart illustrating a method of fabricating a portable non-combustion vaporizer device for generating vapors, in accordance with an embodiment of the present disclosure.

FIG. 19 is a flowchart illustrating a method of generating vapors by way of a portable non-combustion vaporizer device, in accordance with an embodiment of the present disclosure.

Corresponding reference characters indicate corresponding components throughout the several figures of the Drawing. Elements in the several figures are illustrated for simplicity and clarity and have not necessarily been drawn to scale. For example, the dimensions of some of the elements in the figures may be emphasized relative to other elements for facilitating understanding of the various presently disclosed embodiments. Also, common, but well-understood, elements that are useful or necessary in commercially feasible embodiment are often not depicted in order to facilitate a less obstructed view of these various embodiments of the present disclosure.

### DETAILED DESCRIPTION

The following description is not to be taken in a limiting sense, but is made merely for the purpose of describing the general principles of exemplary embodiments. The scope of the disclosure should be determined with reference to the Claims. Reference throughout this specification to “one embodiment,” “an embodiment,” or similar language denotes that a particular, feature, structure, or characteristic, described in connection with the embodiment, is included in at least one embodiment of the present disclosure. Thus, appearances of the phrases “in one embodiment,” “in an embodiment,” and similar language throughout this specification may, but do not necessarily, all refer to the same embodiment.

Further, the described features, structures, or characteristics of the present disclosure may be combined in any suitable manner in one or more embodiments. In the Detailed Description numerous specific details are provided for a thorough understanding of embodiments of the disclosure. One skilled in the relevant art will recognize, however, that the embodiments of the present disclosure can be practiced without one or more of the specific details, or with other methods, components, materials, and so forth, and still remain encompassed by the present disclosure. In other instances, well-known structures, materials, or operations are not shown or described in detail to avoid obscuring aspects of the present disclosure.

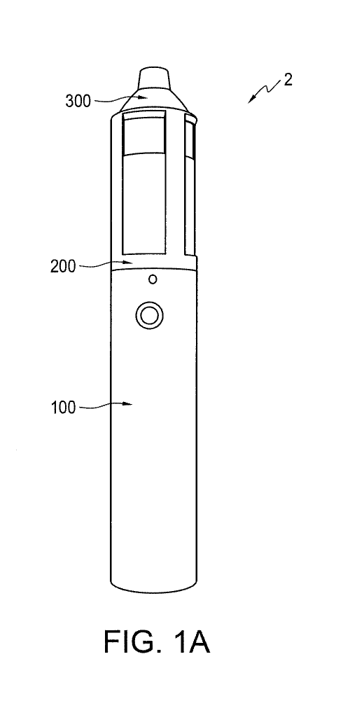

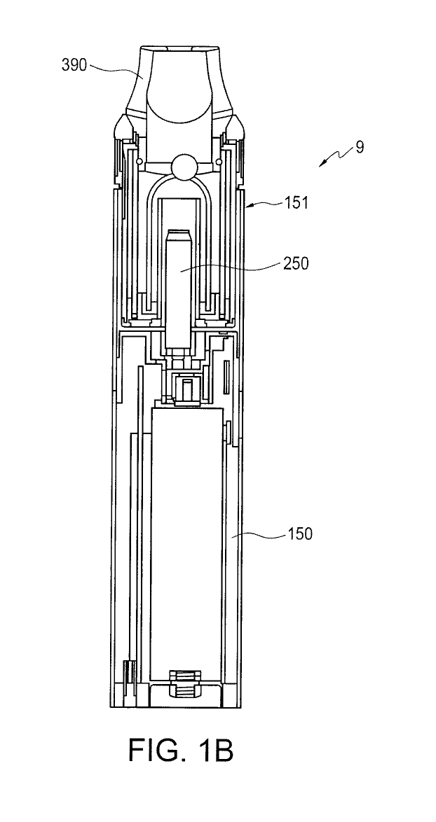

Referring to FIG. 1A, this diagram illustrates, in a front elevation perspective view, a vaporizer device 2, such as would be included in a vaporizer apparatus, in accordance with an embodiment of the present disclosure. The vaporizer 2 comprises a heating component 100, a protective collar 200, and a cartridge 300. In some embodiments, the protective collar 200 retains the cartridge 300 against a portion of the heating component 100, such as to enable heating of a vaporizable substance held within a vaporization chamber within the cartridge 300, to produce a vapor which is inhalable or consumable by a user. The heating component 100 is adapted to couple with the protective collar 200; and the protective collar 200 is adapted to couple with the cartridge 300.

Referring now to FIG. 1B, this diagram illustrates, in a cut-away front elevation perspective view, a vaporizer device 9, such as would be included in a vaporizer apparatus, in accordance with a further embodiment of the present disclosure. The vaporizer 9 comprises a heating component base or handle 150, a heating element 250, and a cartridge 390 adapted for fitting within an open top collar or flange 151 of heating component base 150. In some embodiments, the top collar or flange 151 of heating component base 150 retains the cartridge 390 against a portion of the heating component 150, such as against an internal heating element 250 for example, such as to enable heating of a vaporizable substance held within a vaporization chamber within the cartridge 390, and to produce a vapor which is inhalable or consumable by a user. The top collar or flange 151 of heating component base 150 is adapted to couple with the cartridge 390. In some embodiments, heating component base 150 may desirably be adapted for handheld use by a user and may be configured in a shape suitable for holding in one or both hands of the user, such as by grasping the outside cylindrical surface of the heating component base 150.

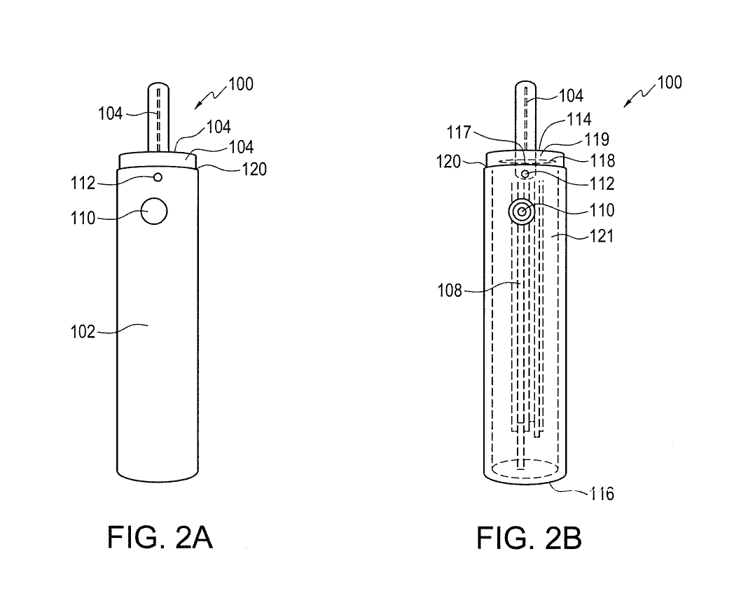

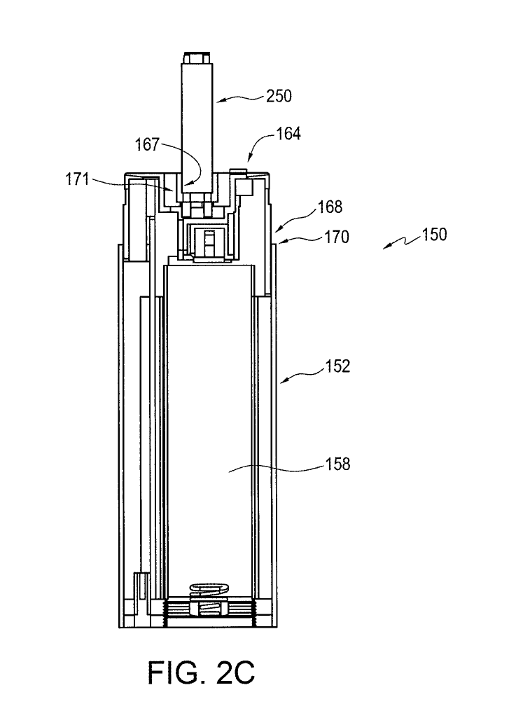

Referring to FIG. 2A, this diagram illustrates, in a front elevation perspective view, a heating component 100, in accordance with an embodiment of the present disclosure. The heating component 100 comprises a case 102, a heating element 104, circuitry 108 (as shown in FIG. 2B), and a power switch 110. The heating element 104 is coupled with the circuitry 108, such as by electrical connection. Circuitry 108, operated by the power switch 110, controls the amount of electrical current that is provided to the heating element 104, which in one embodiment may comprise one or more electrical filaments which may be resistively heated by the passage of electrical current therethrough. In some embodiments, the power switch 110 enables controlling (adjusting), e.g., by a user, the amount of current that is provided to the heating element 104, such as to control or adjust the temperature or heat produced by the heating element 104, and which may desirably control the extent or amount of vaporization of a vaporizable substance held within a connected cartridge when attached to the heating component 100. In one such embodiment, the power switch 110 may comprises one of a digital switch and an analog switch. In one embodiment, the heating element 104 is set to a temperature of approximately 450° F. when activated. In other embodiments, the heating element 104 may emit heat comprising a temperature ranging from approximately 300° F. to approximately 500° F. when activated. The power switch 110 is coupled with a power source (not shown), such as a battery, an uninterrupted power supply, and a power outlet, such as for connection to mains electrical power for operation when plugged in to “the grid” for example.

Still referring to FIG. 2A, the heating element 104 may comprise any suitable heat-emitting device, such as that which converts electricity into heat through the process of resistive or joule heating. In some embodiments, the heating element 104 may comprise at least one of a nichrome (nickel-chromium alloy) or tungsten heating element, a resistance wire, an etched foil, a radiative heating element, a conductive grid or mesh heating element, and the like. In some embodiments, the heating element 104 may be encased in a thermally conductive material, such as glass, plastic, metal ceramic, and the like. In a particular embodiment, the heating element 104 may comprise a heat-emitting filament that is encased in glass, such as a light bulb.

Referring to FIG. 2B, this diagram illustrates, in a front elevation perspective view, the heating component 100, as shown in FIG. 2A, with an outer surface shown as transparent for furthering understanding of any internal components, in accordance with an embodiment of the present disclosure. The heating component 100 comprises an outer case 102, a heating element 104, circuitry 108, and a switch 110. The heating component 100 further comprises a top 114 and a bottom 116. The top 114 of the case 102 comprises an aperture 117 for receiving the heating element 104. In this embodiment of the present disclosure, the aperture 117 is substantially centrally located in relation to the top 114. In some embodiments of the present disclosure, the aperture 117 is located in a non-central position, that is, the aperture 117 may be located closer to one side of the top 114.

Still referring to FIG. 2B, in a particular illustrative embodiment, the heating element 104 comprises a light bulb that is insertable in relation to the aperture 117 of the case 102 and is configured to electrically couple with the circuitry 108. The heating element 104 extends from the top 114 of the case 102. The heating element 104 extends substantially perpendicularly from the top 114 of the case 102. In some embodiments, a plurality of apertures 117 are disposable in relation to the top 114 and are configured to receive a plurality of heating elements 104. Other configurations of the aperture 117 in relation to the top 114 of the case 102 are possible and encompassed by the present disclosure. In some embodiments of the present disclosure, the case 102 comprises an air conduit 121. In other embodiments, a portion of case 102 comprises an air conduit 121. In yet other embodiments of the present disclosure, a tube or channel (not shown) is insertable within the case 102 to act as the air conduit 121.

Still referring to FIG. 2B, the top 114 comprises at least one air aperture 119 that facilitates air flow therethrough, e.g., air flow from the top 114 of the case 102 into, and out of, the air conduit 121. The case 102 further comprises at least one additional air aperture 112 that facilitates air flow through sides of the case 102 into, and out of, the air conduit 121. Accordingly, air may flow through the case 102 by entering the at least one air aperture 112 through the air conduit 121 and exiting the at least one air aperture 119. In some embodiments of the present disclosure, the at least one air aperture 112 may be located in relation to other portions of the case 102, such as the bottom 116 of the case 102, wherein the air conduit 121 connects the at least one air aperture 112 to the at least one air aperture 119 disposed in relation to the top 114.

Still referring to FIG. 2B, in some embodiments of the present disclosure, the at least one air aperture 112 may not be required, such as in an embodiment wherein a plurality of air gaps (not shown) are disposable in relation to the case 102 which also facilitate sufficient air flow to enter the air conduit 121. In some embodiments of the present disclosure, the case 102 comprises at least one air permeable material. In some embodiments of the present disclosure, the top 114 comprises an air-permeable material, such as at least one of a mesh, a woven material, an interlaced material, and a vapor-permeable fabric, such as cotton or polyester. In these embodiments of the present disclosure, the at least one air aperture 119 may not be required, whereby the air-permeable material facilitates air to flow from the air conduit 121.

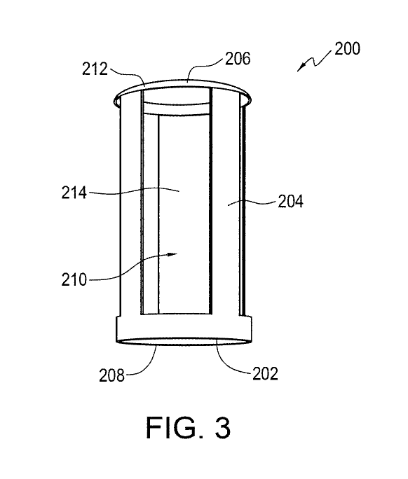

Still referring to FIG. 2B, in one embodiment, the case 102 comprises a recessed surface 118 disposed relative to the body of the case 102 for facilitating removable coupling, such as with a collar 200 or a cartridge 300, for example. The recessed surface 118 extends from an edge of the top 114 of the case 102 to a ridge 120 (also see FIG. 2A). An outer portion of the recessed surface 118, having substantially constant outer dimension(s), is configured, e.g., sized, to removably couple with an inner portion of a bore 210, having inner dimension(s), in the collar 200 (see FIG. 3). The inner dimension(s) of bore 210 approximate the substantially constant outer dimension(s) of the recessed surface 118 for a distance corresponding to the axial length of the recessed surface 118, wherein the substantially constant outer dimension(s) of the recessed surface 118 comprises a range that is equal to, or less than, e.g., slightly less than, that of the bore 210. In some embodiments of the present disclosure, the substantially constant outer dimension(s) of the recessed surface 118 comprises a range that is less than, e.g., slightly less than, approximately 0.01 mm to approximately 3 mm, e.g., corresponding to approximately 1/256 inch to approximately ⅛ inch of the dimension of bore 210.

Referring now to FIG. 2C, this diagram illustrates, in a cut-away front elevation perspective view, a heating component base or handle 150, comprising a heating element 250, in accordance with another embodiment of the present disclosure. The heating component base 150 comprises an outer case 152, a heating element 250, circuitry 158, and a switch operable by a user (not shown). The heating component base 150 further comprises a top 164 and a bottom 166. The top 164 of the case 152 comprises an aperture 167 for receiving the heating element 250. In this embodiment of the present disclosure, the aperture 167 is substantially centrally located in relation to the top 164. In some embodiments of the present disclosure, the aperture 167 may alternatively be located in a non-central position, that is, the aperture 167 may be located closer to one side of the top 164.

Still referring to FIG. 2C, the heating element 250 is operably coupled with the circuitry 158, such as by electrical connection. Circuitry 158, operated by one or more signal from a user, such as operation of a power switch (not shown) for example, controls the amount of electrical current that is provided to the heating element 250, which in one embodiment may comprise one or more electrical filaments which may be resistively heated by the passage of electrical current therethrough. In some embodiments, the operation of the heating component base 150 enables controlling (adjusting), e.g., by a user, the amount of current that is provided to the heating element 250, such as to control or adjust the temperature or heat produced by the heating element 250, and which may desirably control the extent or amount of vaporization of a vaporizable substance held within a connected cartridge when attached to the heating component 150. In one such embodiment, similar to as shown in the embodiment depicted in FIG. 2A, a user-operable power switch may comprises one of a digital switch and an analog switch. In one such embodiment, the heating element 250 may be set to a temperature of approximately 450° F. when activated. In other embodiments, the heating element 250 may emit heat comprising a temperature ranging from approximately 300° F. to approximately 500° F. when activated. In one embodiment, the circuitry 158 may comprise a power source (not shown), such as a battery, an uninterrupted power supply, and a power outlet, such as for connection to mains electrical power for operation when plugged in to “the grid” for example. In one such embodiment, similar to as shown in FIG. 2A, the heating element 250 may comprise any suitable heat-emitting device, such as that which converts electricity into heat through the process of resistive or joule heating. In some embodiments, the heating element 250 may comprise at least one of a nichrome (nickel-chromium alloy) or tungsten heating element, a resistance wire, an etched foil, a radiative heating element, a conductive grid or mesh heating element, and the like. In some embodiments, the heating element 250 may be encased in a thermally conductive material, such as glass, plastic, metal ceramic, and the like. In a particular embodiment, the heating element 150 may comprise a heat-emitting filament that is encased in a substantially cylindrical glass surface, such as a light bulb, or other protective glass heat conductive shield, for example.

Still referring to FIG. 2C, in a particular illustrative embodiment, the heating element 250 is insertable in relation to the aperture 167 of the case 152 and is configured to electrically couple with the circuitry 158 such as to provide for electrically powered heating operation of the heating element 250. In such a case, the heating element 250 extends from the top 164 of the case 152. Substantially similar to the embodiment as shown in FIG. 2A, in some embodiments of the present disclosure, heating element 250 may be alternately located and configured, and may comprise more than one heating element component (not shown). In one embodiment, the case 152 may comprise an air conduit (not shown). In another embodiment, the case 152 may comprise a plurality of air gaps (not shown) which may facilitate sufficient air flow to enter the case 152 such that air may exit from one or more air conduits 171 which may be disposed in or near the top 164 of the case 152, such as to allow for air to enter into the base of a cartridge (not shown) engaged with the top 164 of the case 152 during use, for example. In some embodiments of the present disclosure, the case 152 comprises at least one air permeable material. In some embodiments of the present disclosure, the top 164 comprises an air-permeable material, such as at least one of a mesh, a woven material, an interlaced material, and a vapor-permeable fabric, such as cotton or polyester, such as may provide for allowing air flow into and/or out of a cartridge (not shown) engaged with the heating component base 150 during use.

Still referring to FIG. 2C, in one embodiment, the case 152 comprises a recessed surface 168 disposed relative to the body of the case 152 for facilitating removable coupling, such as with a collar 200 or a cartridge 390, for example. The recessed surface 168 extends from an edge of the top 164 of the case 152 to a ridge 170. An outer portion of the recessed surface 168, having substantially constant outer dimension(s), may desirably be configured, e.g., sized, to removably couple with an inner portion of a bore 210 of a collar 200 (see FIG. 3), or alternatively to removably couple with an inner portion of a bore of a cartridge 390, or further alternatively to allow coupling with a top collar or flange 151 of heating component base 150, such as to retain a cartridge 390 against a portion of the heating component 150, such as against an internal heating element 250 for example.

Referring now to FIG. 3, this diagram illustrates, in a front elevation perspective view, a collar 200, in accordance with an embodiment of the present disclosure. The collar 200 is substantially tubular and comprises an inner surface 202, an outer surface 204, a top 206, and a bottom 208. The inner surface 202 defines a bore 210. The bore 210 extends from the top 206 of the collar 200 to the bottom 208 of collar 200. The bore 210 is sized to receive a cartridge 300. In some embodiments, the cartridge 300 is adapted to slidably insert into the bore 210 of the collar 200 from the bottom 208.

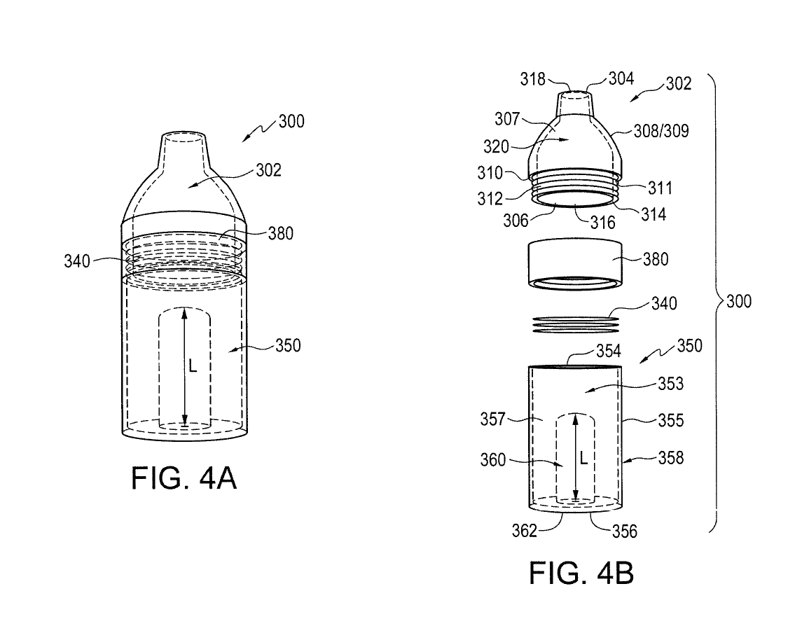

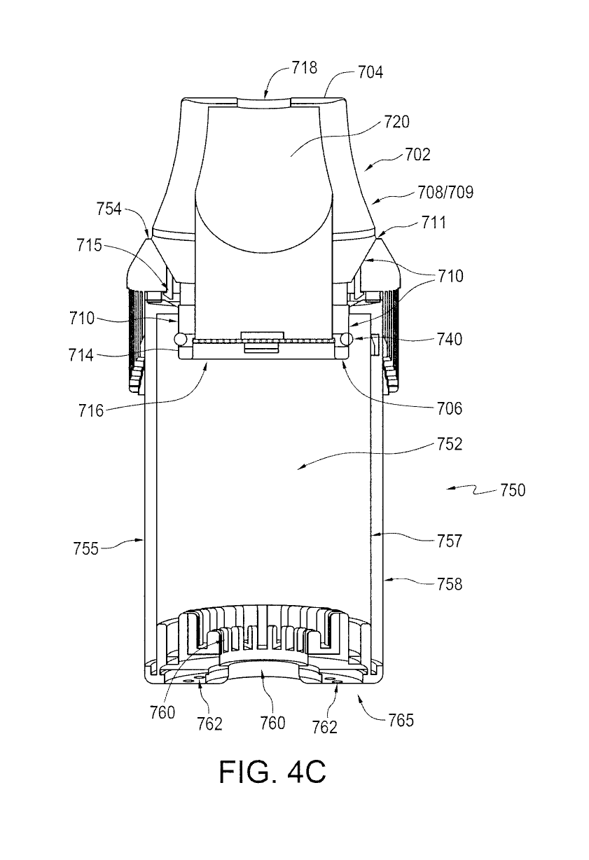

Still referring to FIG. 3, in one embodiment, the collar 200 is connectable in relation to the case 102. In one embodiment, the bottom 208 of the collar 200 is disposed over the top 114 of the case 102. In this embodiment, a proximal end of the bottom 208 abuts the ridge 120 of the case 102, while a portion of the inner surface 202 is adjacent the recessed surface 118. The collar 200 is further configured to removably couple with the heating component 100 (or the case 102). The collar 200 protects the heating element 104 from physical damage when the collar 200 is coupled with the heating component 100. The collar 200 also protects a user from heat emitted from the heating element 104 when the heating element 104 is activated. In some embodiments, the collar 200, through thermal conduction, facilitates reducing the temperature of vapors produced in a vaporization chamber within the cartridge 300 (see FIGS. 4A, 4B and 4C), e.g., vapors of at least one consumable substance held within the cartridge 300.

Still referring to FIG. 3 and referring back to FIGS. 2A and 2B, in one embodiment, to removably couple the collar 200 with the case 102, at least one structure for attachment (not shown) may be used. In one embodiment, at least one seal (not shown), such as a sealable gasket, an O-ring, a washer, and the like, is disposable in relation to the recessed surface 118 (FIGS. 2A and 2B). In this embodiment of the present disclosure, the recessed surface 118 comprises at least one seal-seat (not shown), such as an O-ring receptacle, sized to receive the at least one seal, such as an O-ring (not shown). When the collar 200 is attached to the case 102, the at least one seal is disposable between the collar 200 and the case 102, and facilitates frictionally coupling the collar 200 with the case 102. The collar 200 is removable from the case 102 by way of applying a sufficient axial force thereto. In other embodiments of the present disclosure, other various suitable connection techniques, such as a pressure-fit, an interference-fit, a snap fit and a magnet-retained releasable fit are employable for facilitating removably coupling the collar 200 with the case 102.

Still referring to FIG. 3 and referring back to FIGS. 2A and 2B, in some embodiments of the present disclosure, the collar 200 may be removably coupled with the case 102 such as by way of complementary threads (not shown) respectively located on the recessed surface 118 and the inner surface 202. In some embodiments of the present disclosure, complementary magnets (not shown) are insertable into the collar 200 and the case 102. In some embodiments of the present disclosure, a complementary latch-and-anchor system (not shown) may be used for coupling the case 102 with the collar 200. The collar 200 further comprises a flange or lip 212 located at the top 206 of the collar 200. The lip 212 has an inner dimension(s) that is less than the inner dimension(s) of the bore 210 and less than the maximum outer dimension(s) of the cartridge 300. The lip 212 inner dimension(s) being smaller than the bore 210 inner dimension(s) is, thereby, adapted to retain the cartridge 300 within the bore 210 of the collar 200 when the cartridge 300 is inserted through the bottom 208.

Still referring to FIG. 3 and referring to back to FIG. 1, the collar 200 is configured to couple with the case 102, either with or without the cartridge 300 being inserted through the bore 210. In embodiments of the present disclosure, wherein a cartridge 300 is disposed in the collar 200, the collar 200 substantially rigidly retains, e.g., holds, cartridge 300 between the lip 212 and the top 114 of the case 102. In one embodiment, the collar 200 desirably comprises at least one opening or window 214 for facilitating viewing cartridge 300 and/or the heating element 104. The window 214 permits a user to view the vaporization process and/or the light emitted from the heating element 104, if any. In a further alternative embodiment, such as for use with the heating component base 150 illustrated in FIGS. 1B and 2C, the heating component base 150 may comprise an integrated top collar or flange 151 of heating component base 150 which is operable to retain the cartridge 390 against a portion of the heating component 150, such as against an internal heating element 250 for example.

Referring to FIG. 4A, this diagram illustrates a front elevation perspective view of a cartridge 300, with an outer surface shown as transparent for furthering understanding of any internal components, in accordance with an embodiment of the present disclosure. The cartridge 300 comprises a mouthpiece 302 and a vaporization chamber 350. In some embodiments of the present disclosure, the cartridge 300 further comprises at least one seal 340, such as a sealable gasket, an O-ring, a washer, and a tamper seal 380, and the like.

Referring to FIG. 4B, this diagram illustrates, in an exploded front elevation perspective view, the cartridge 300, as shown in FIG. 4A, with the outer surface shown as transparent for furthering understanding of any internal components, in accordance with an embodiment of the present disclosure. The mouthpiece 302 comprises a top 304, a bottom 306, an inner surface 307, and an outer surface 308. As shown in FIG. 4B, the inner surface 307 comprises a shape having a reducing dimension along its longitudinal axis, such as a conical, or funnel, shape, thereby forming a funnel-shaped bore 320 along such longitudinal axis. The inner surface 307 is further defined by a surface of a vapor-permeable base 314 opposing the top 304. The bore 320 comprises a top opening 318 located at the top 304. Although the bore 320 is shown as being substantially circular by example only, it may comprise any cross-sectional shape having a vapor-permeable base 314 and a top opening 318. The top opening 318 is narrower in relation to the bottom 306.

Referring to FIG. 4C, this diagram illustrates, in a cut-away front elevation perspective view, a cartridge or pod 700 according to a further embodiment of the present disclosure. In one such embodiment, cartridge or pod 700 comprises a mouthpiece 702 comprising a top 704, a top opening 718, and a bottom 706, and a vaporization chamber 750 comprising a top 754 and bottom 756, and inside surface 757 and outside surface 758. In some embodiments of the present disclosure, the cartridge 700 further comprises at least one seal 740, such as a sealable gasket, an O-ring, a washer, and a tamper seal, or the like.

Referring to FIGS. 4B and 4C, the top opening 318 comprises a shape and size to comfortably insert into a user's mouth. In one such embodiment, the top opening 318, 718 of cartridge 300, 700 may comprise at least one of a substantially circular or oval cross-sectional shape, for example. In other embodiments, the top opening 318, 718 may be shown as being circular by example only and may comprise any suitable cross-sectional shape, such as a circle, half circle, polygon, moon-shaped, and the like. In some embodiments of the present disclosure, the cross-sectional area of opening 318, 718 comprises a range of approximately 0.75 cm² to approximately 20 cm². In other embodiments of the present disclosure, the cross-sectional area of top opening 318, 718 comprises a range of approximately 1.75 cm² to approximately 7 cm². In some embodiments of the present disclosure, the mouthpiece 302, 702 is adapted to couple with at least one feature, such as other mouthpieces, hoses, tubes, whips, conduits, adapters, and the like, e.g., by way of configuring the opening 318, 718. In some embodiments of the present disclosure, the mouthpiece 302, 702 comprises a plurality of openings (not shown) to allow concurrent use, e.g. by a plurality of users.

Still referring to FIGS. 4B and 4C, the base 314, 714 of the mouthpiece 302, 702 of cartridge 300, 700 prevents herbs and other consumable substances from entering, e.g., accidentally, the bore 320, 720. In one embodiment, the mouthpiece 302, 702 may comprise a single vapor-impermeable material. That is, the mouthpiece 302, 702 and the base 314, 714 may comprise a single material and/or may be integrally formed. In a particular embodiment of the present disclosure, the base 314, 714 comprises at least one aperture 316, 716. The at least one aperture 316, 716 allows the passage of vapor, located outside the mouthpiece 302, 702, such as from the vaporization chamber 350, 750, to enter the bore 320, 720 of mouthpiece 302, 702, and then to exit the opening 318, 718.

Still referring to FIGS. 4B and 4C, in some embodiments of the present disclosure, the base 314, 714 may comprise a material distinct from the remaining portions of mouthpiece 302, 702. In these embodiments of the present disclosure, the base 314, 714 may comprise at least one of a mesh, a woven material, an interlaced material, and a vapor-permeable fabric, such as cotton or polyester. In some embodiments of the present disclosure, the base 314, 714 may comprise a liner (not shown). The liner may be attached to a top surface or a bottom surface of the base 314, 714. The liner may comprise at least one of a mesh, a woven material, an interlaced material, and a vapor-permeable fabric, such as cotton or polyester.

Still referring to FIGS. 4B and 4C, in some embodiments of the present disclosure, the mouthpiece 302, 702 may desirably be disposable and replaceable, e.g., such as when permeability of the vapor-permeability base 314, 714 decreases due to a buildup of vaporization residue, when refilling of vaporization chamber 350, 750 is required, or to avoid transfer of taste or smell of a consumable substance to another incompatible substance. In some embodiments of the present disclosure, the mouthpiece 302, 702 may comprise at least one of glass, ceramic, plastic, metal, and any other suitable material. In some embodiments, the mouthpiece 302, 702 comprises a recyclable material. In an embodiment of the present disclosure, the mouthpiece 302, 702 comprises an outer surface 308, 708, the outer surface 308, 708 comprising an upper surface 309, 709 and a recessed surface 310, 710. The upper surface 309, 709 is the portion of the outer surface 308, 708 extending from the opening 318, 718 to the ridge 311, 711. The recessed surface 310, 710 is defined as a portion of the outer surface 308, 708 extending from the ridge 311, 711 to the edge of the base 314, 714.

Still referring to FIGS. 4B and 4C, the upper surface 309, 709 optionally comprises gradually reducing cross-section dimension(s), with a maximum dimension located approximately at the ridge 311, 711 and a minimum dimension located approximately at the top opening 318, 718. Reiterating, in one embodiment, the mouthpiece 302 of the cartridge 300 is insertable into the bore 210 of the collar 200 for removable coupling with the heating component 100. In one such embodiment of the present disclosure, the outer surface 308 is sized to facilitate insertion of the mouthpiece 302 into the bore 210. Accordingly, the maximum outer dimension(s) of the outer surface 308 of the mouthpiece 302 comprises a range that approximates that of the inner dimension(s) of the bore 210. The mouthpiece 302 is retained within the bore 210 of the collar 200 by the lip 212.

In another embodiment, the dimension of the outer surface 758 of cartridge 700 may desirably be sized to facilitate insertion into a top flange or retaining ring 151 of a heating component base 150, such as to provide for removable and interchangeable use of a heating component base 150 with one or more cartridges 700, such as may comprise one or more desired consumable substance, for example. Similarly, in another such embodiment, the dimension of the vaporization chamber 750 at its top ridge 711, and the mouthpiece 702 at its maximum dimension near ridge 711 may desirably be sized to facilitate insertion of the mouthpiece 702 into the bore of vaporization chamber 750, such as after filling of the chamber 750 with a consumable substance in which case the mouthpiece 702 may be lockably or releaseably coupled to the chamber 750 to allow for one-time filling or re-filling of the chamber 750 respectively. In an alternative embodiment, mouthpiece 702 may be releaseably coupled to the vaporization chamber 750 such as to allow for replacement of the mouthpiece 702 for use with multiple users, or for vaporization of multiple different consumable substances, such as to reduce contamination or transfer of flavors of residue from one user to another or from use with one consumable substance to another, for example.

Referring to FIG. 4B and referring back to FIG. 3, in an embodiment of the present disclosure, the top 304 of the mouth piece 302 is insertable through the bottom 208 of the collar 200. As the mouthpiece 302 reaches the top 206 of the collar 200, a portion of the mouthpiece 302, such as the outer surface 308, extends beyond the lip 212. In some embodiments, the mouthpiece 302 does not entirely exit the bore 210 of the collar 200. At least a portion of the outer surface 308 of the mouthpiece 302 abuts and is retained by the lip 212 of the collar 200. In some embodiments, the inner dimension(s) of the lip 212 are approximately equal to that of the outer dimension(s) of the mouthpiece 302 at the top opening 318. In those embodiments of the present disclosure, the mouthpiece 302 is entirely retainable within the collar 200.

Still referring to FIG. 4B and FIG. 4C, the mouthpiece 302, 702 is configured to couple with the vaporization chamber 350, 750. In an embodiment of the present disclosure, the outer dimension(s) of the recessed surface 310, 710 comprise a range that is approximately equal to, or less than, the inner dimension(s) of a bore 352, 752 of the vaporization chamber 350, such as a vaporization chamber 350, 750 suitable to contain at least one vaporizable consumable substance (such as but not limited to including one or more of liquid, herbal, dried plant, tobacco, or cannabis substances, for example), wherein the mouthpiece 302, 702 is insertable into the bore 352, 752 of the chamber 350, 750. When the bottom 306, 706 of the mouthpiece 302, 702 is inserted into the bore 352, 752 of the chamber 350, 750, the ridge 311, 711 of the mouthpiece 302, 702 may desirably abut a top 354 754 edge of the chamber 350, 750. In one embodiment, the mouthpiece 702 may lockably couple with the chamber 750, such as by engagement of one or more locking clips, catches, snaps or other suitable lockably engageable retaining means 715.

Still referring to FIG. 4B and FIG. 4C, in some embodiments of the present disclosure, the mouthpiece 302, 702 may be removably coupled with the chamber 350, 750. In some embodiments of the present disclosure, wherein the mouthpiece 302, 702 is coupled with the chamber 350, 750, a seal is formed between the mouthpiece 302, 702 and the chamber 350, 750. In an embodiment of the present disclosure, the recessed surface 310, 710 comprises at least one seal receptacle 312, e.g., at least one O-ring receptacle, that is sized to receive at least one seal 340, 740, such as an O-ring. The at least one seal 340, 740 may typically be disposed around a recessed surface (such as surface 309) in the at least one seal receptacle 312.

Still referring to FIG. 4B and FIG. 4C, when the mouthpiece 302, 702 is inserted into the chamber 350, 750, the recessed surface 310, 710 is adjacent an inner surface of the chamber 350, 750; and the at least one seal 340, 740 may be positioned between the adjacent surfaces of the mouthpiece 302, 702 and the chamber 350, 750, thereby forming a vapor seal, and/or in some embodiments thereby frictionally engaging the mouthpiece 302, 702 with the chamber 350, 750. In one embodiment, the mouthpiece 302, 702 may be removable from the chamber 350, 750 by exerting a sufficient axial force thereto, and/or by disengaging one or more mechanical locking catches or snaps, such as catches 715, for example. In other embodiments of the present disclosure, various other techniques, such as a pressure-fit technique and an interference-fit technique, are employable for removably coupling the mouthpiece 302, 702 to the chamber 350, 750.

Still referring to FIG. 4B and FIG. 4C, and referring back to FIG. 4A, in some embodiments of the present disclosure, the mouthpiece 302, 702 may be substantially permanently or permanently attached to, lockably coupled to, or integrally formed with, the chamber 350, 750. In certain such embodiments of the present disclosure, an adhesive, such as at least one of a polymer, a glue, any chemical bonding agent, and any mechanical bonding agent, is applicable between the recessed surface 310, 710 and the chamber 350, 750 for bonding the mouthpiece 302, 702 with the chamber 350, 750, or a suitable mechanical locking means such as one or more locking clips, catches, snaps or the like (such as catches 715 for example) may be used to substantially lockingly couple mouthpiece 302, 702 with chamber 350, 750. In some embodiments of the present disclosure, an outer seal 380 may be employed to substantially permanently couple the chamber 350 with the mouthpiece 302. In a particular embodiment of the present disclosure, an outer seal 380 may comprises at least one of an adhesive foil, an adhesive paper, an adhesive plastic, and other suitable material. In some embodiments, an outer seal 380 may comprise at least one of branding information, product warnings, tamper resistant indicators, and other security features. In some embodiments, an outer seal 380 may comprise a barcode, a printable electronic memory device, a radio-frequency identification (RFID) device or other suitable readable memory device or label which may comprise stored information transmittable to circuitry 108. Information may include, but is not limited to, at least one of vaporization temperature, product information, security verification, authentication verification, expiry date, and other relevant information. In another embodiment, a further seal may be placed over the top or tip 304, 704 of the cartridge 300, 700, such as to prevent contamination or tampering or to ensure hygienic storage of the mouthpiece 302, 702 of the cartridge 300, 700 until opened by a user, for example.

In another embodiment, a readable electronic label or chip memory device (not shown), such as a printable thin film memory device (such as Thin Film printable memory devices available from the Xerox™ Corporation, for example), or an RFID (radio frequency identification) device or the like, containing information regarding the at least one consumable substance that is predisposed in the chamber 350, 750, may be attached to an outer surface of the cartridge 300, 700, such as to a surface of the vaporization chamber 350, 750 and/or the mouthpiece 302, 702, for example. In one such embodiment, the circuitry 108, 158 comprises a compatible electronic memory reader such as a thin film memory reader or RFID reader or the like, for retrieving the information emitted by the memory device comprised in or attached to the cartridge 300, 700 and setting the heating element 104, 154 temperature or other operational settings or variables to the desired temperature or setting, such as to provide desired operation or vaporization characteristics for a particular consumable substance.

Still referring to FIG. 4B and FIG. 4C, the chamber 350, 750 comprises a base 356, 756 and a shell 355, 755. The base 356, 756 is disposable in relation to the shell 355, 755, thereby leaving an opposing open top 354, 754. The shell 355, 755 has an inner surface 357, 757 and outer surfaces 358, 758. The inner surface 357, 757 of the shell 355, 755 defines a portion of the bore 352, 752 extending from a top opening 354, 754 to the base 356, 756. The bore 352, 752 is sized to accommodate or store at least one consumable substance, e.g. herbal materials, tobacco, liquid and/or cannabis materials, for vaporization. The base 356, 756 is configured to receive the heating element 104, 154. The base 356, 756 comprises a heating receptacle 360, 760, such as a vaulted internal portion or a domed internal portion, which comprise an inner substantially cylindrical surface of the chamber 350, 750, and define a substantially annular portion of chamber 350, 750 between the inner surface 357, 757 of the shell 355, 755, and the heating receptacle surface 360, 760. The heating receptacle 360, 760 extends a length L (not shown in FIG. 4C), toward the opening 354, 754, the length L comprising a range that is approximately equal to, or greater than, the portion of the heating element 104, 154 extending perpendicularly from the top 114 of heating component 100. When the chamber 350, 750 is disposed over the heating element 104, 154, the heating receptacle 360, 760 surrounds the top and side portions of the heating element 104, 154. In some embodiments of the present disclosure, the surface of the heating receptacle 360, 760 abuts the heating element 104, 154.

Still referring to FIG. 4B and FIG. 4C, in various embodiments of the present disclosure, when the heating element 104, 154 is operating, heat may be transferred through the heating receptacle 360, 760 by at least one of conduction and radiation. The heating receptacle 360, 760 transfers heat to the at least one consumable substance disposed in the bore 352, 752 by at least one of further conduction and further radiation. In some embodiments of the present disclosure, the heating receptacle 360, 760 transfers heat to the base 356, 756 by at least conduction. In some of these embodiments of the present disclosure, the base 356, 756 transfers heat to the shell 355, 755 by at least conduction. In various embodiments of the present disclosure, heat is transferable towards the at least one consumable substance by at least one of the heating receptacle 360, 760, the base 356, 756, and the shell 355, 755.

Still referring to FIG. 4B and FIG. 4C, the base 356, 756 comprises at least one aperture 362, 762. The aperture 362, 762 facilitates air flow into the bore 352, 752, e.g., to “carburate” the at least one consumable substance. In one such embodiment, the heat transfer from the heating element 104, 154 to the consumable substance may further also optionally comprise convection such as by transfer of air heated by the heating element 104, 154 into the bore 352, 752 to additionally provide heat for the purposes of vaporization of the comsumable substance. In some embodiments, the base 356, 756 comprises a material that is distinct from the remaining portion of the chamber 350, 750. In certain such embodiments of the present disclosure, the base 356, 756 may comprise at least one of a mesh, a woven material, an interlaced material, and a vapor-permeable fabric, such as cotton or polyester. In some alternative embodiments of the present disclosure, the base 356, 756 may comprise a liner (not shown), the liner comprising at least one of a mesh, a woven material, an interlaced material, and a vapor-permeable fabric, such as cotton or polyester.

Still referring to FIG. 4B and FIG. 4C, and also referring back to FIG. 4A, in an embodiment of the present disclosure, the cartridge 300, 700 comprises a disposable cartridge (which may also be referred to as a pod), wherein the cartridge 300, 700 further comprises a mouthpiece 302, 702, a chamber 350, 750, and at least one consumable substance, and wherein the at least one consumable substance is predisposed or pre-filled in the bore 352, 752 of the chamber 350, 750. The mouthpiece 302, 702 is then substantially permanently, sealably, attached to the chamber 350, 750 via at least one of a seal 340, 740, such as a sealable gasket, an O-ring, a washer, a locking or snap-locking mechanical closure, catch or clip (such as catch 715), and a tamper seal 380, and the like, optionally also in conjunction with an adhesive (not shown), such as at least one of a polymer, a glue, any chemical bonding agent, and any mechanical bonding agent. In one such embodiment, a vendor of pre-filled cartridges or pods 300, 700 may thereby pre-fill and seal a consumable substance in the bore 352, 752 of the chamber 350, 750 which may be securely retained therein safe from tampering for delivery to a consumer, such as for providing assurance of quality or safety from a known vendor or other source, for example.

Still referring to FIG. 4B and referring back to FIGS. 1A and 2B, in some embodiments of the present disclosure, the case 102 and the chamber 350, together, form a complementary key-and-notch system to align (register) the aperture 362 with the air aperture 119, located on the top 114 of the case 102. In an embodiment of the present disclosure, a protrusion (not shown), or a key, extends perpendicularly from the top 114. A corresponding notch (not shown) is disposable in relation to the base 356, wherein the base 356 abuts the top 114 of the case 102 only when the key and notch of the key-and-notch system (not shown) are engaged.

Still referring to FIG. 4B and FIG. 4C, and also referring back to FIGS. 1A, 1B and 2B, in some embodiments of the present disclosure, the at least one consumable substance in the cartridge 300, 700 comprises a desired, or an optimum, vaporizing temperature, wherein the at least one consumable substance is predisposed. The cartridge 300, 700 further comprises a feature (not shown) for communicating with the circuitry 108, 158 to control the temperature of the heating element 104, 154. For example, the communicating feature may comprise a readable electronic label or chip memory device, such as a printable thin film memory device (such as Thin Film printable memory devices available from the Xerox™ Corporation, for example), or an RFID (radio frequency identification) device or the like, containing information regarding the at least one consumable substance that is predisposed in the chamber 350, 750. In one embodiment, the circuitry 108, 158 comprises a compatible electronic memory reader such as a thin film memory reader or RFID reader or the like, for retrieving the information emitted by the memory device comprised in or attached to the cartridge 300, 700 and setting the heating element 104, 154 temperature or other operational settings or variables to the desired temperature or setting, such as to provide desired operation or vaporization characteristics for a particular consumable substance. In a further embodiment, the communicating feature may additionally comprise other wired and wireless communication features, such as Bluetooth® or other radio communication capability, a barcode scanning capability, and the like. In a further embodiment, circuitry 108, 158 may further comprise communication features to allow for communication with one or more other electronic devices, such as a mobile phone or computer and the like, such as for providing information regarding a consumable substance in a particular cartridge or pod 300, 700, or details regarding users, consumption, quantities remaining, usage tracking and/or vaporizer device operation or troubleshooting, for example.

Still referring to FIG. 4B and referring back to FIG. 1A, in an embodiment of the present disclosure, a vaporizer device 2 comprises a cartridge 300 and a heating component 100, wherein the cartridge 300 comprises a disposable cartridge or pod. The vaporizer device 2 further comprises a protective collar 200. The cartridge 300 is inserted into the collar 200, such that a lip 212 of collar 200 abuts an outer surface 308 of the cartridge 300. The collar 200, along with the cartridge 300, is removably attachable to the case 102 of the heating component 100. For example, the heating element 104 is inserted into the heating receptacle 360 of the cartridge 300, while a recessed surface 118 of the case 102 is inserted into a bore 210 of the collar 200.

Still referring to FIG. 4B and FIG. 4C, and also referring back to FIG. 1A and FIG. 1B, in an embodiment of the present disclosure, when a user operates a vaporizer device 2, 9, the circuitry 108, 158 within the heating component 100, 150 provides current from a battery (not shown) to the heating element 104, 154 in a time range that is nearly (approximately) instantaneous in reaching a desired, or an optimum, vaporizing temperature. In some embodiments of the present disclosure, the time period comprises a range of less than approximately 5 seconds. In some embodiments of the present disclosure, the time period comprises a range of less than approximately 30 seconds. When the heating element 104, 154 emits heat, the heat is transferred via at least one of conduction, convection, and radiation to the cartridge 300, 700 through at least the heating receptacle 360, 760, and/or the cartridge base 356, 756. In one embodiment, circuitry 108, 158 and heating element 104, 154 may comprise a dual-power heating element system, where one or more heating element 104, 154 may be powered at multiple power levels, such as a high power level and a low power level, for example. In one such embodiment, the circuitry 108, 158 may comprise a first high power heating circuit, operable to provide a first high power level to the one or more heating element 104, 154, such as may be desirable to enable rapid heating of the consumable substance in cartridge 300, 700 to a desired vaporization temperature, for example. In such an embodiment, the circuitry 108, 158 may also comprise a second lower power heating circuit, operable to provide a second lower power level to the one or more heating element 104, 154, such as may be desirable to enable maintenance of a desired vaporization temperature of the consumable substance while consuming less power than the first high power level, for example. In an exemplary such embodiment, a first high power level may comprise about 45-80 W, and more particularly about 60 W, for example, and a second lower power level may comprise about 10-20 W and more particularly about 15 W, for example. In such a manner, in one embodiment, a dual-power heating element system may desirably allow for both rapid initial heating of a consumable substance, and more power efficient maintenance of a desired vaporization temperature after it is initially reached, as may be beneficial in particular for mobile battery powered embodiments of the present disclosure which may desirably be configured to provide enhanced battery life from a limited battery power storage capacity, for example.

Still referring to FIG. 4B and FIG. 4C, and also referring back to FIG. 1A and FIG. 1B, in some embodiments of the present disclosure, the heating receptacle 360, 760 of the cartridge 300, 700 may desirably radiate and conduct heat to the at least one consumable substance such as for heating the substance to a desired vaporization temperature. In some embodiments of the present disclosure, the chamber 350, 750 comprises a thermally conductive material, wherein the entire chamber 350, 750 conducts and radiates heat to the at least one consumable substance. When the at least one consumable substance reaches a desired temperature, or an optimum, vaporizing temperature, active ingredients of the at least one consumable substance boil and vaporize. Suction is applied, e.g., via a user placing his/her mouth on the mouthpiece 302, 702, thereby inducing a negative pressure, or at least a partial vacuum, within the bore 320, 720 of the mouthpiece 302, 702. The negative pressure, in turn, facilitates movement of the vapor collected in the chamber 350, 750 to the user's mouth.

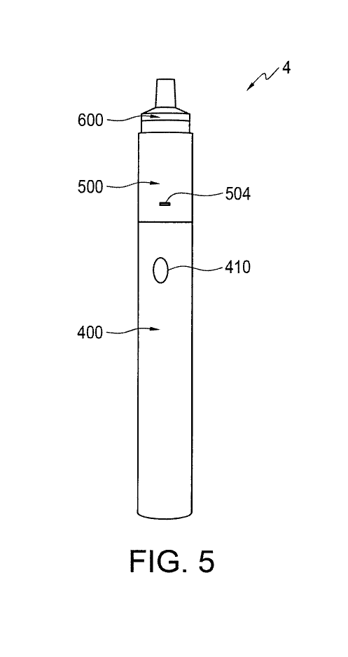

Referring to FIG. 5, this diagram illustrates, in a front elevation perspective view, a vaporizer device 4, such as would be included in a vaporizer apparatus, in accordance with an embodiment of the present disclosure. The vaporizer device 4 comprises a heating component 400, a protective adapter 500, and a cartridge 600. The protective adapter 500 is configured to couple with both the heating component 400 and the cartridge 600.

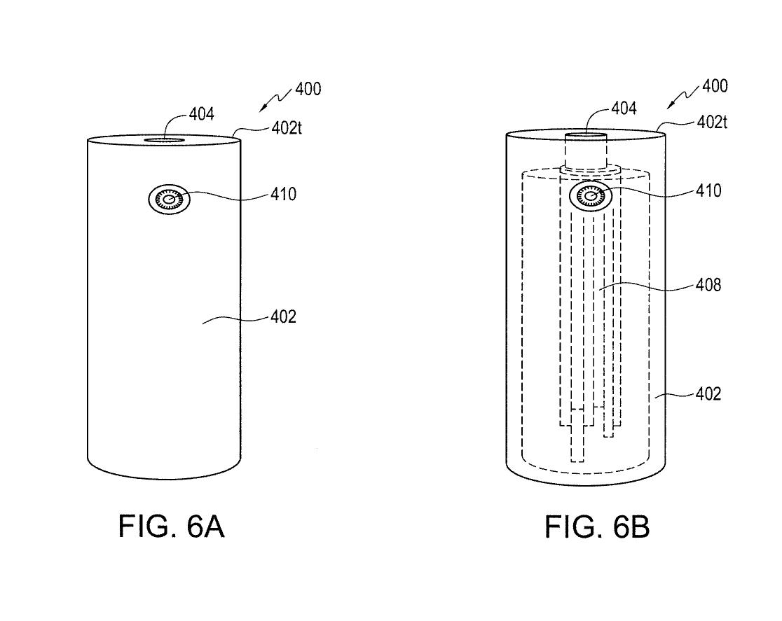

Referring to FIG. 6A, this diagram illustrates, in a front elevation perspective view, the circuitry component 400, as shown in FIG. 5, in accordance with an embodiment of the present disclosure. The circuitry component 400 comprises a case 402, an adapter receptacle 404, and a power source (not shown), such as a battery, circuitry 408 (as shown in FIG. 6B), and a power switch 410. The circuitry component 400 is similar to the heating component 100; however, in this embodiment, the circuitry component 400 comprises an adapter receptacle 404, rather than an aperture 117, for receiving the heating element 104. In some embodiments, the adapter receptacle 404 of the circuitry component 400 is sized for receiving a heating element 104.

Referring to FIG. 6B, this diagram illustrates, in a front elevation perspective view, the circuitry component 400, as shown in FIG. 6A, with the outer surface shown as transparent for further understanding of internal components, in accordance with an embodiment of the present disclosure. The case 402 is similar to the case 102, a power button 410 is similar to the power button 110; and circuitry 408 is similar to circuitry 108, as shown in FIG. 1. In some embodiments, the circuitry component 400 comprises a power source (not shown), such as a battery component. The circuitry component 400 comprises a power source, such as batteries, e.g., rechargeable batteries (such as any known rechargeable battery type including but not limited to lithium, lithium-ion, lithium-polymer, nickel metal hydride, nickel-cadmium batteries, and the like), single use batteries such as alkaline, lithium or other suitable battery types, such as known rechargeable and non-rechargeable batteries operable for handheld heating devices such as e-cigarette batteries, vaporizer batteries, and the like.

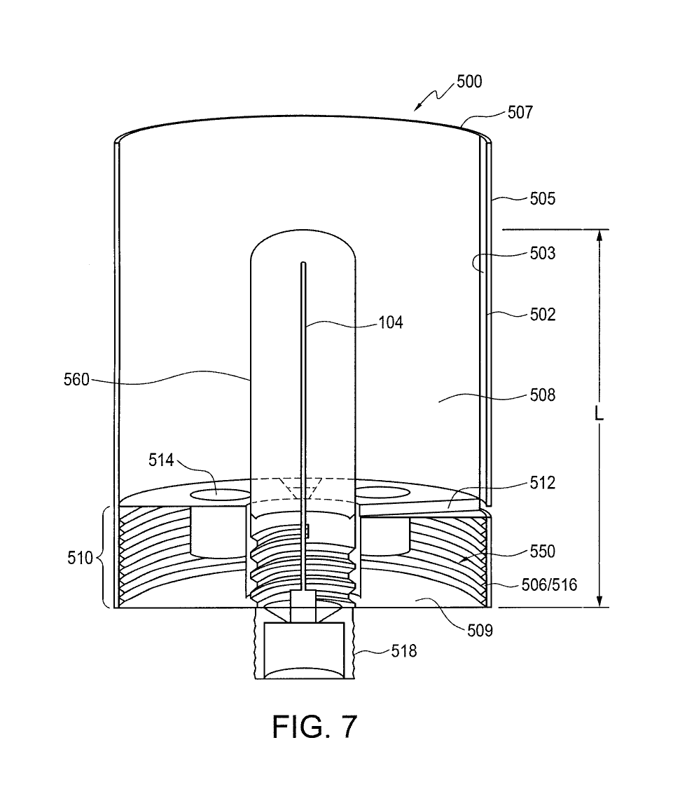

Still referring to FIG. 6B, the adapter receptacle 404 is configured to receive an adapter 500 (FIG. 7). The adapter 500 comprises a connector portion 518 that is insertable into the adapter receptacle 404 and is configured to electrically couple with circuitry 408. In some embodiments, the adapter receptacle 404 is sized as a “510” connector. In other embodiments, the adapter receptacle 404 is sized to accommodate at least one connector type, such as a “401” type, a “510” type, a “901” type, an “E9” type, and a “4081” type, and the like. In some embodiments, the adapter receptacle 404 is fixably attachable to the adapter 500. In some embodiments, the adapter 500 is fixably attached to the adapter receptacle 404 by complementary threads.

Referring to FIG. 7, this diagram illustrates, in a cut-away front elevation perspective view, an adapter 500, such as a protective adapter, in accordance with an embodiment of the present disclosure. The protective adapter 500 comprises a protective shell 502, a heating element 104, 154, and an adapter 510. The shell 502 has an inner surface 503 and an outer surface 505. The inner surface 503 of the shell 505 defines a portion of a bore 508 extending from a top opening 507 to a bottom opening 509. The bore 508 is sized to accommodate or store at least one consumable substance, e.g., herbal, tobacco, e-liquid (such as glycol based liquid) materials, and the like, for vaporization.

Still referring to FIG. 7, the protective adapter 500 is shown here engaged with an adapter 510 (FIG. 8), wherein the adapter 510 comprises a top 511. The adapter 510 is configured to receive an exemplary heating element 104, 154. The adapter 510 comprises a heating receptacle 560, such as a vaulted portion or a domed portion. The heating receptacle 560 extends a length L, toward the top opening 507, the length L comprising a range that is approximately equal to, or greater than, the portion of the heating element 104, 154 extending perpendicularly from the top 511.

Still referring to FIG. 7, when the protective adapter 500 is disposed over the heating element 104, 154, the heating receptacle 560 surrounds the top and side portions of the heating element 104, 154. In some embodiments of the present disclosure, the surface of the heating receptacle 560 abuts the heating element 104, 154. The shell 502 comprises a lower portion 550 having threads 506 for engaging complementary threads 516 of the adapter 510 (FIG. 8).

Referring to FIG. 8, this diagram illustrates, in a front elevation perspective view, an adapter 510, having a heating element 104, 154 disposed therethrough, in accordance with an embodiment of the present disclosure. The adapter 510 comprises a top 511 and a bottom 513. The adapter 510 is configured to receive the heating element 104, 154. The adapter 510 comprises a heating receptacle 560, such as a vaulted portion or a domed portion. The heating receptacle 560 extends a length L, toward the top opening 507, the length L comprising a range that is approximately equal to, or greater than, the portion of the heating element 104, 154 extending perpendicularly from the top 511. The top 511 comprises at least one channel 512 and at least one magnet 514. The adapter 510 comprises threads 516 for engaging complementary threads 506 of shell 502.

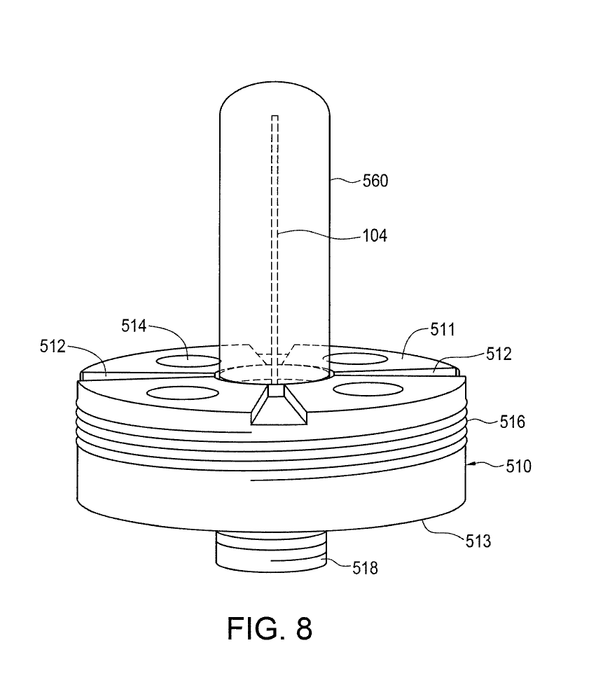

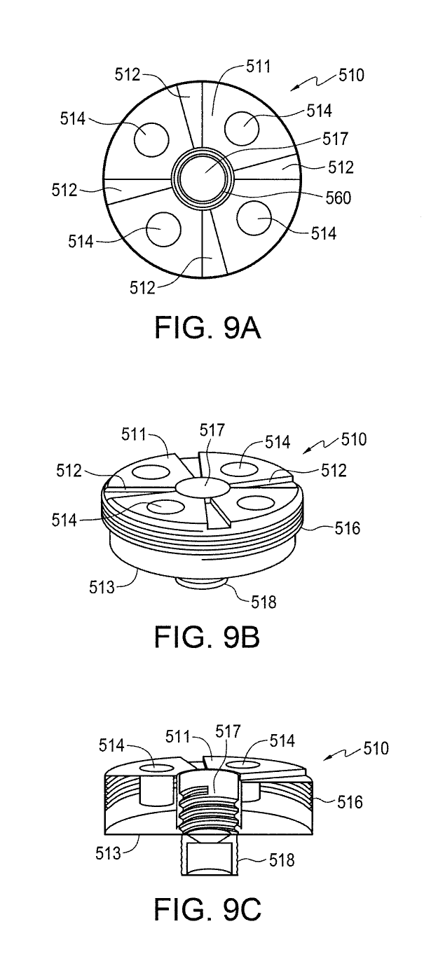

Referring to FIG. 9A, this diagram illustrates, in a top view, an adapter 510, in accordance with an embodiment of the present disclosure. The adapter 510 comprises a substantially disk-shape, having a top 511 and a bottom 513 (See also FIGS. 9B and 9C). The top 511 comprises at least one channel 512, at least one magnet 514, and an aperture 517 for accommodating a heating element 104, 154. The adapter 510 is configured to receive the heating element 104, 154. The adapter 510 comprises a heating receptacle 560, such as a vaulted portion or a domed portion. The heating receptacle 560 extends a length L, toward the top opening 507, the length L comprising a range that is approximately equal to, or greater than, the portion of a heating element 104, 154 extending perpendicularly from the top 511.

Referring to FIG. 9B, this diagram illustrates, in a front elevation perspective view, the adapter 510, as shown in FIG. 9A, in accordance with an embodiment of the present disclosure. The adapter 510 is disposable in relation to a circuitry component 400. In one embodiment, the adapter 510 comprises a connector portion 518 extending perpendicularly from the bottom 513 of the adapter 510. The connector portion 518 is complementarily sized to an adapter receptacle 404 (As shown in FIGS. 6A and 6B). In this embodiment, the connector portion 518 is sized for compatibility with at least one of connector type of a “401” type, a “510” type, a “901” type, an “E9” type, and a “4081” type, and the like.

Still referring to FIG. 9B, in this embodiment, when the adapter 510 is disposed in the circuitry component 400, the adapter 510 and circuitry component 400 are electrically connected. The top 511 of the adapter 510 comprises an aperture 517 for receiving ae heating element 104, 154. In this embodiment of the present disclosure, the aperture 517 is positioned at a central position in relation to the top 511. In some embodiments of the present disclosure, the aperture 517 is disposed in a non-central region, e.g., the aperture 517 is proximally disposable in relation to one side of top 511.

Referring to FIG. 9C, this diagram illustrates, in a cut-away front elevation perspective view, the adapter of 510, as shown in FIG. 9A, in accordance with an embodiment of the present disclosure. The bottom 513 abuts the top 402t of the case 402 when the connector portion 518 is electrically coupled with the adapter receptacle 404. In an embodiment, the connector portion 518 and the adapter receptacle 404 comprise complementary threaded portions for facilitating both an electrical coupling and a physical coupling. The adapter 510, having an outer edge, comprises at least one air channel 512 extending radially inward from the outer edge toward the aperture 517. In some embodiments, the at least one air channel 512 extends from the outer edge of the adapter 510 to the outer edge of the aperture 517.

Still referring to FIG. 9C and referring back to FIGS. 9A, 9B, and 5, the adapter 510 is configured to removably attach to a cartridge 600. In some embodiments, the adapter 510 comprises at least one magnet 514. The at least one magnet 514 is molded or affixed in relation to the top 511 of the adapter 510. In some embodiments, the at least one magnet 514 is disposed between the at least two air channels 512. In some embodiments, the adapter 510 comprises a magnetic material.

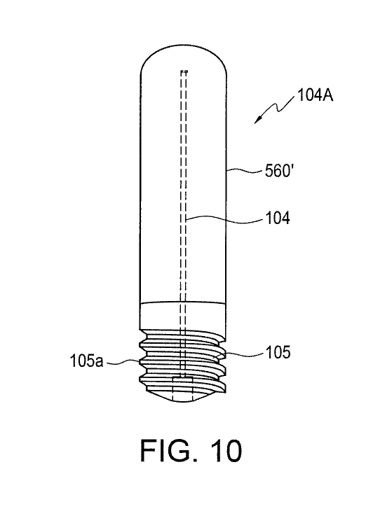

Referring to FIG. 10 and referring back to FIG. 2, this diagram illustrates, in a front view, a heating element 104, 154, in accordance with an embodiment of the present disclosure. The heating element 104, 154 comprises any heat-emitting device capable of converting electricity into heat, e.g., through the process of resistive or joule heating. In some embodiments, the heating element 104, 154 comprises at least one of a nichrome wire, a resistance wire, an etched foil, a radiative heating element, and the like. In some embodiments, the heating element 104, 154 is encased or encapsulated in housing 560′, wherein the housing 560′ comprises a thermally conductive material, yet electrically insulative material, such as glass, a polymer, a non-electrically conducting polymer, a plastic, a ceramic, a composite material, non-electrically conducting composite material, and the like.

Still referring to FIG. 10 and referring back to FIG. 2, in an embodiment, the heating element 104, 154 comprises a heat-emitting filament encased or encapsulated in a housing 560′, wherein the housing 560′ comprises a glass material, such as in a light bulb heating element configuration 104A, by example only. The heating element 104, 154 further comprises a connector portion 105 configured to electrically couple with the apertures 117, 517 (FIG. 2B). In some embodiments, the connector portion 105 and the apertures 117, 517 comprise a plurality of electrically conductive threads 105a to facilitate electrically coupling, wherein the plurality of electrically conductive threads 105a comprises at least one material, such as a metal and an alloy.

Still referring to FIG. 10 and referring back FIG. 8, the exemplary light bulb configuration 104A is insertable into the aperture 517 of the adapter 510. Heating element 104, 154 extends from the top 511 of the adapter 510. The heating element 104, 154 extends substantially perpendicularly from the top 511 of adapter 510. In some embodiments, a plurality of apertures 517 is disposed in relation to the top 511 for receiving a plurality of heating elements 104, 154, such as embodied in light bulb configurations 104A. Other configurations of the plurality of apertures 517 are possible and encompassed by the present disclosure.

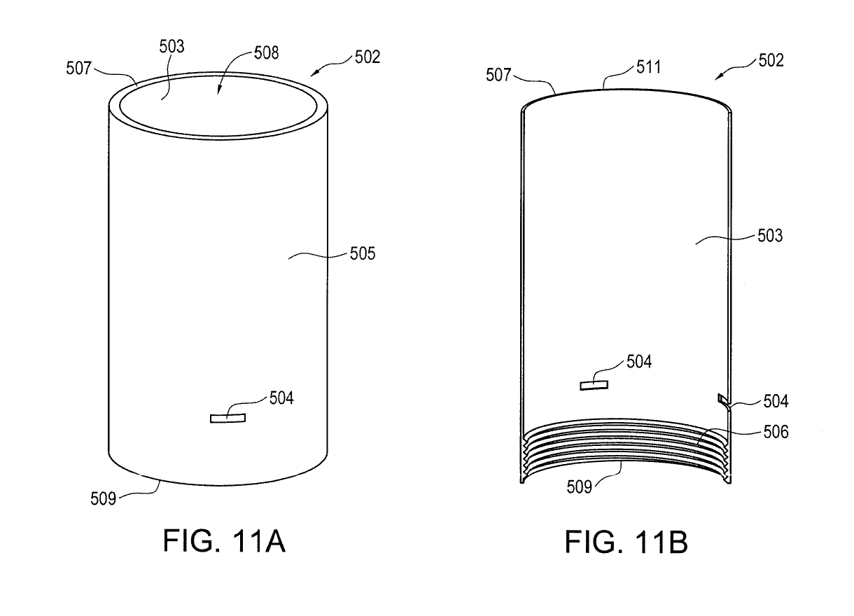

Referring to FIG. 11A and referring back to FIG. 5, this diagram illustrates, in a front elevation perspective view, a protective shell 502, in accordance with an embodiment of the present disclosure. The protective shell 502 comprises a substantially tubular configuration, by example only, having an inner surface 503 (see FIG. 11B), an outer surface 505, a top 507, and a bottom 509. The inner surface 503 defines a bore 508 that extends from the top 507 of the shell 502 to the bottom 509 of shell 502. The bore 508 is sized to receive a cartridge 600 (FIG. 5). In some embodiments, the cartridge 600 is adapted to slidably insert into the bore 508 of shell 502 from the top 507.

Referring to FIG. 11B, this diagram illustrates, in a front elevation cut-away view, the protective shell 502, as shown in FIG. 11A, in accordance with an embodiment of the present disclosure. The shell 502 comprises at least one ridge 506 protruding from the inner surface 503 of the shell 502 along the bottom 509. In this embodiment, when the bottom 509 of the shell 502 is coupled with the adapter 510, at least one seal 516, such as an O-ring, rests below, or between, the at least one ridge 506. The at least one ridge 506 provides frictional forces, whereby an axial force is required to remove the shell 502 from the adapter 510, whereby inadvertent removal of the shell 502 from the adapter 510 is minimized.

Still referring to FIG. 11B and referring back to FIGS. 7 and 11A, the shell 502 is disposable in relation to the adapter 510. In an embodiment, the bottom 509 of the shell 502 is disposed over the top 511 of adapter 510. In an embodiment, the shell 502 is configured to removably couple with the adapter 510, wherein at least one structure for attachment may be used. In an embodiment, at least one seal, such as a sealable gasket, an O-ring, and the like, is disposable in relation to the outer surface 516 of the adapter 510. In some embodiments, the outer surface 516 comprises at least one receptacle (not shown), such as an O-ring seat, sized to receive the at least one seal, such as an O-ring. When the bottom 509 of the shell 502 is attached to the adapter 510, the at least one seal is disposed between the shell 502 and the adapter 510, thereby frictionally holding or retaining the shell 502 in relation to the case 102. The shell 502 is removable from the adapter 510 by way of a sufficient applied axial force.

Still referring to FIG. 11B and referring back to FIG. 7, in some embodiments of the present disclosure, the shell 502 is removably attachable to the adapter 510 by way of complementary threads respectively located on the outer surface 516 of the adapter 510 and the inner surface 506 of the shell 502. In some embodiments of the present disclosure, complementary magnets (not shown) are respectively insertable into the adapter 510 and the shell 502. In some embodiments of the present disclosure, a complementary latch-and-anchor system (not shown) may be used for coupling the shell 502 with the adapter 510. In some embodiments of the present disclosure, the shell 502 is permanently attached to the adapter 510. In these embodiments of the present disclosure, an adhesive, such as at least one of a polymer, a glue, any chemical bonding agent, and any mechanical bonding agent, is applicable between the outer surface 516 of the adapter 510 and the inner surface 506 of the shell 502 to bond the shell 502 together with the adapter 510.

Still referring to FIG. 11B and referring back to FIGS. 11A and 7, in some embodiments, the shell 502 comprises at least one aperture 504, such as through-aperture, disposed between the outer surface 505 and the inner surface 503. When the shell 502 is attached to the adapter 510, the aperture 504 facilitates ventilation, e.g., the transfer of air from outside the shell 502 to the inside the shell 502, and vice versa. In some embodiments, when the shell 502 is attached to the adapter 510, the aperture 504 is aligned with the channel 512 of the adapter 510 (see FIG. 7). In this embodiment, the channel 512 acts as an air conduit when the bore 508 contains a physical object, such as a cartridge 600. In this embodiment, the channel 512 and the aperture 504, at least, facilitates the transfer of air from outside shell 502 to an area proximal the heating element 104.

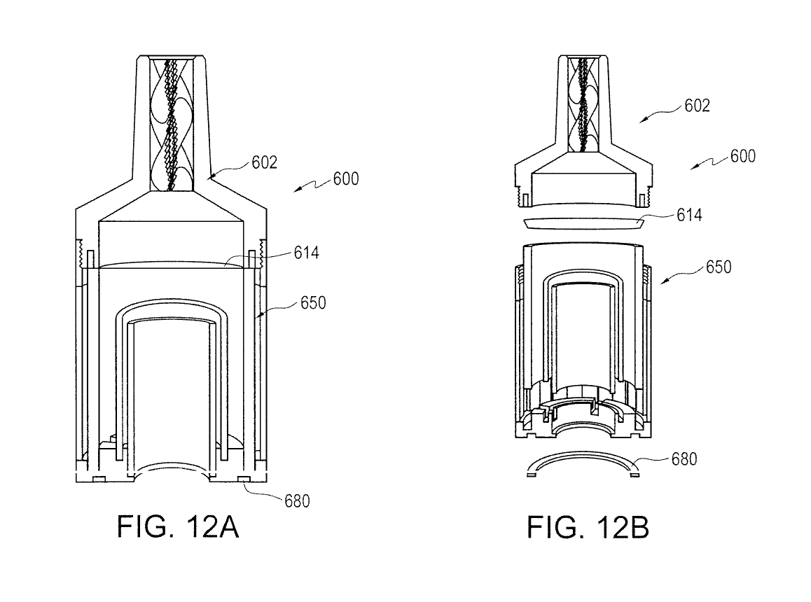

Referring to FIG. 12A and referring back to FIG. 5, this diagram illustrates, in a cut-away front elevation perspective view, a cartridge 600, in accordance with an embodiment of the present disclosure. In some embodiments of the present disclosure, the at least one consumable substance disposed in the cartridge 600 comprises a desired or optimum vaporizing temperature. The cartridge 600 further comprises a feature (not shown) for communicating with circuitry 408 to control the temperature of heating element 104. For example, the communicating feature may comprises an electronically readable memory storage device, such as a printed memory label, an RFID device, or other suitable memory device, which may contain information regarding the at least one consumable substance predisposed in the chamber 673. The circuitry 408 may comprise a compatible memory reading device, such as a printed memory label reader, or RFID reader for retrieving the information emitted by the memory device of the cartridge 600 and desirably may further be operable to set the heating element 104, 154 temperature to the desired temperature (set-point). In some embodiments, the communicating feature may additionally comprise one or more wired and/or wireless communication features, such as Bluetooth®, barcode scanning, and the like. In a further embodiment, circuitry 408 may further comprise communication features to allow for communication with one or more other electronic devices, such as a mobile phone or computer and the like, such as for providing information regarding a consumable substance in a particular cartridge or pod 600, or details regarding users, consumption, quantities remaining, usage tracking and/or vaporizer device operation or troubleshooting, for example.

Referring to FIG. 12B and referring back to FIG. 5, this diagram illustrates, in an exploded cut-away front elevation perspective view, the cartridge 600, as shown in FIG. 12A, comprising a mouthpiece 602 and a vaporization chamber 650. In an embodiment of the present disclosure, a vaporizer device 4 comprises a disposable cartridge 600, an adapter 500, and a heating component 400. In one such embodiment, the adapter 500 is attachable to the heating component 400 through complementary threaded portions. A disposable cartridge 600 is insertable into the adapter 500, and removably retained by complementary magnets 514 and a retaining member 680, the retaining member comprising at least one material, such as a metal and an alloy.

Referring to FIG. 12B and referring back to FIGS. 12A and 5, in one embodiment of the present disclosure, when a user operates a vaporizer device 4, the circuitry 408 within the heating component 400 provides current from a power source, such as a battery (not shown) to the heating element 104, 154 in a time period that is nearly (approximately) instantaneous in reaching a desired temperature. In some embodiments of the present disclosure, the time period comprises a range of less than approximately 5 seconds. In some embodiments of the present disclosure, the time period comprises a range of less than approximately 30 seconds. When the heating element 104, 154 emits heat, the heat is transferred via at least one of conduction, convection, and radiation to the cartridge 600. When the at least one consumable substance reaches a desired temperature, the active ingredients of the at least one consumable substance boil and vaporize. Suction is applied, e.g., via a user placing his/her mouth on the mouthpiece 602, thereby inducing a negative pressure or at least a partial vacuum within the bore 620 of the mouthpiece 602. The negative pressure, in turn, facilitates movement of the vapor collected in chamber 650 to the user's mouth. In one embodiment, similar to as described above in relation to FIGS. 4A and 4B, circuitry 408 and heating element 104, 154 may comprise a dual-power heating element system, where one or more heating element 104, 154 may be powered at multiple power levels, such as a high power level and a low power level, for example.

Referring to FIGS. 12A and 12B and referring back to FIG. 5, in some embodiments of the present disclosure, circuitry 108, 408 may communicate via Bluetooth® or other communication techniques, e.g., wireless techniques, to an electronic device for providing information. Information may include cartridge information, usage information, battery information, and the like. In some embodiments of the present disclosure, the vaporizer device 2, 9 or 4 comprises a non-portable vaporizer (not shown). The non-portable vaporizer comprises a plurality of multiple heating components for receiving multiple cartridges, wherein multiple users may share a single vaporizer device 2, 9 or 4, having a plurality of distinct cartridges 300, 700, 600 attached. In some embodiments of the present disclosure, the vaporizer device 2, 9 or 4 comprises a wired battery connection for use with an electrical outlet on mains or grid electrical power.

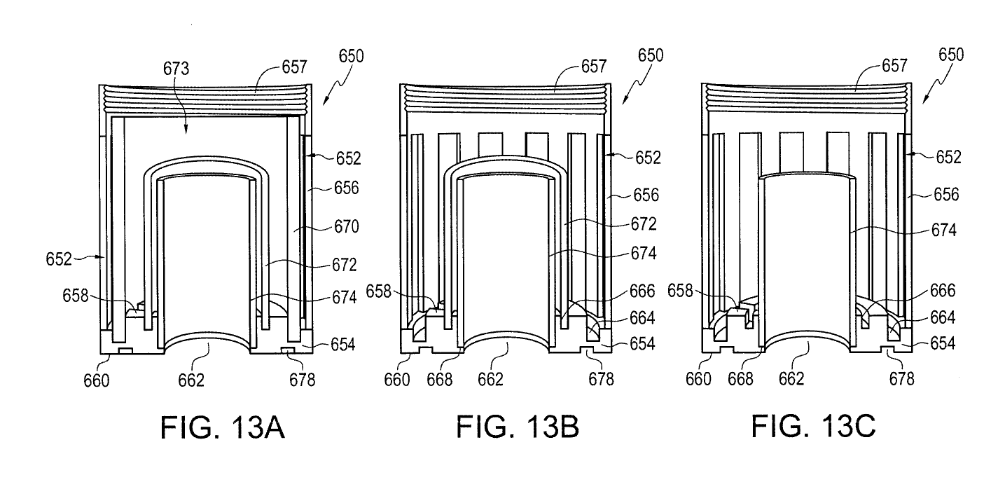

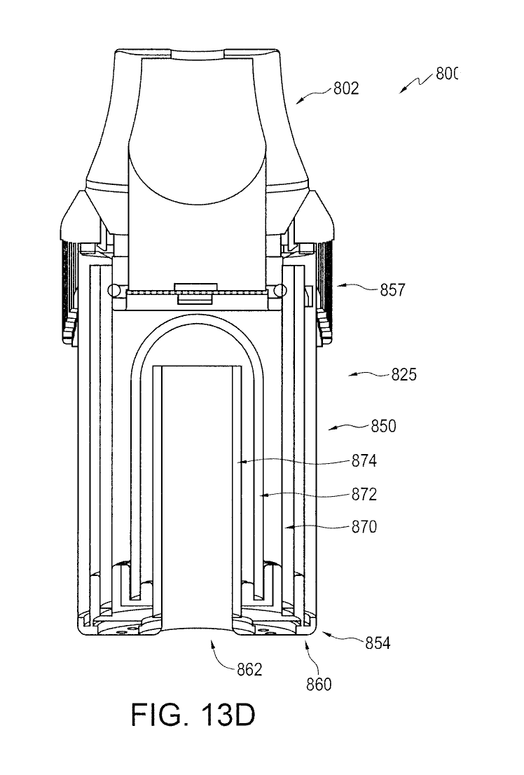

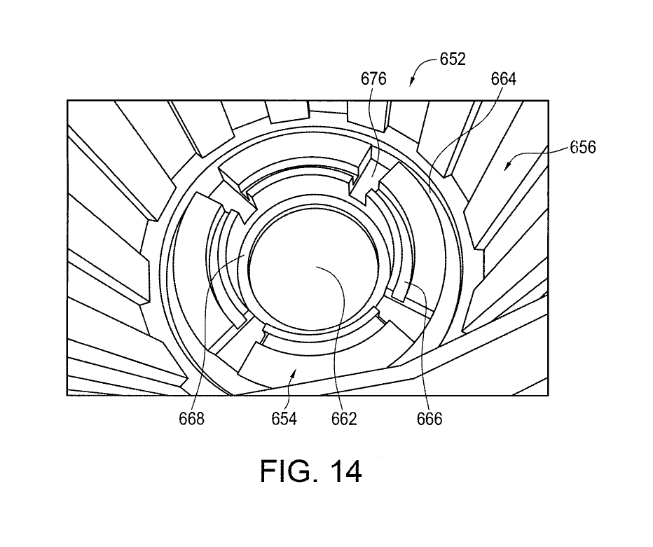

Referring to FIG. 13A, this diagram illustrates, in a cut-away front elevation perspective view, a vaporization chamber 650, in accordance with an embodiment of the present disclosure. The chamber 650 comprises a protective shell 652, an outer wall or ring 670, an inner wall or ring 672, and a heating receptacle wall or ring 674. In one embodiment, one or more of the outer wall or ring 670, the inner wall or ring 672, and the heating receptacle wall or ring 674 comprise a suitably heat conductive material such as glass, ceramic, metal, polymer or composite that are suitable to conduct heat by conduction and/or radiation from a heating element (not shown) inserted into the heating receptacle wall or ring 670, such as for heating a consumable substance contained in the vaporization chamber 650 between the outer and inner walls or rings 670, 672. The shell 652 comprises a base portion 654 and a guard portion 656. The base portion 654 has a top surface 658, and bottom surface 660. The base portion 654 is configured to receive a heating element, such as exemplary heating element 104, 154. In an embodiment, the base portion 654 is substantially disk-shaped and has an aperture 662 extending from the bottom surface 660 to the top surface 658. The aperture 662 is sized to receive heating element 104. That is, a proximal end of the heating element 104 may be inserted through the aperture 662. In one embodiment, at least one portion of the protective shell 652, base portion 654 and guard portion 656 may desirably comprise a suitable heat resistant and preferably also a suitably heat insulating material such as plastic, glass, polymer, composite or the like, as may be suitable to desirably provide for reducing a temperature of the outer surface of protective shell 652 as may be desired for allowing manual removal or manipulation of the vaporization chamber 650 by a user during and/or following heating. In one such embodiment, the protective shell 652 may desirably reduce the risk of burning to a user handling the vaporization chamber 650 during or following heating, so as to allow for manual removal, exchange or adjustment of the vaporization chamber 650 in a vaporizing device 2, 4, 9, for example.

Referring to FIG. 13B, this diagram illustrates, in a cut-away front elevation perspective view, the chamber 650, as shown in FIG. 13A, with the outer ring 670 removed with the outer surface shown as transparent for furthering understanding of any internal components, in accordance with an embodiment of the present disclosure. The chamber 650 comprises a protective shell 652, an outer ring 670, a heating receptacle ring 672, and an inner ring 674. The shell 652 comprises a base portion 654 and a guard portion 656. The base portion 654 has a top surface 658, and bottom surface 660. The base portion 654 is configured to receive a heating element such as exemplary heating element 104, 154. In an embodiment, the base portion 654 is substantially disk-shaped and has an aperture 662 extending from the bottom surface 660 to the top surface 658. The aperture 662 is sized to receive heating element 104. That is, a proximal end of a heating element 104, 154 may be inserted through the aperture 662.

Referring to FIG. 13C, this diagram illustrates, in a cut-away front elevation perspective view, the chamber 650, as shown in FIG. 13A, with the outer ring 670 and middle heating receptacle 672 removed with the outer surface shown as transparent for furthering understanding of any internal components, in accordance with an embodiment of the present disclosure. The chamber 650 comprises a protective shell 652, an outer ring 670, a heating receptacle ring 672, and an inner ring 674. The shell 652 comprises a base portion 654 and a guard portion 656. The base portion 654 has a top surface 658, and bottom surface 660. The base portion 654 is configured to receive heating element 104. In an embodiment, the base portion 654 is substantially disk-shaped and has an aperture 662 extending from the bottom surface 660 to the top surface 658. The aperture 662 is sized to receive a heating element, such as exemplary heating element 104, 154. That is, a proximal end of a heating element 104, 154 may be inserted through the aperture 662.

Referring to FIG. 13D, this diagram illustrates, in a cut-away front elevation perspective view, a vaporization cartridge 800, comprising a vaporization chamber 850, and a mouthpiece 802, in accordance with an embodiment of the present disclosure. Similar to as described above in reference to FIGS. 13A, 13B and 13C, the vaporization chamber 850 of cartridge 800 comprises a protective shell 852, an outer wall or ring 870, an inner wall or ring 872, and a heating receptacle wall or ring 874. In one embodiment, one or more of the outer wall or ring 870, the inner wall or ring 872, and the heating receptacle wall or ring 874 comprise a suitably heat conductive material such as glass, ceramic, metal, polymer or composite that are suitable to conduct heat by conduction and/or radiation from a heating element (not shown) inserted into the heating receptacle wall or ring 870, such as for heating a consumable substance contained in the vaporization chamber 850 between the outer and inner walls or rings 870, 872. The protective shell 852 comprises a base portion 854, a guard portion 856, and an upper rim portion 857. The base portion 854 has a top surface 858, and bottom surface 860. The base portion 854 is configured to receive a heating element, such as exemplary heating element 104, 154. In an embodiment, the base portion 854 may be substantially disk-shaped and has an aperture 862 extending from the bottom surface 860 to the top surface 858. The aperture 862 is sized to receive an exemplary heating element 104, 154. That is, a proximal end of a heating element 104, 154 may be inserted through the aperture 862. In one embodiment, at least one portion of the protective shell 852, base portion 854, guard portion 856, and upper rim portion 857, may desirably comprise a suitable heat resistant and preferably also a suitably heat insulating material such as plastic, glass, polymer, composite or the like, as may be suitable to desirably provide for reducing a temperature of the outer surface of protective shell 852 as may be desired for allowing manual removal or manipulation of the vaporization chamber 850 by a user during and/or following heating. In one such embodiment, the protective shell 852 may desirably reduce the risk of burning to a user handling the vaporization chamber 850 during or following heating, so as to allow for manual removal, exchange or adjustment of the vaporization chamber 850 in a vaporizing device 2, 4, 9, for example.

Referring to FIG. 14, and also to FIGS. 13A, 13B, 13C and 13D, this diagram illustrates, in a top perspective view, an exemplary protective shell 652 having a base portion 654 and guard portion 656, in accordance with an embodiment of the disclosure. The base portion 654 comprises a first wall or ring channel 664, a second wall or ring channel 666, and a lip or ridge 668 (See also FIGS. 13A-13D). The first wall or ring channel 664 is the outer most g channel relative to the second wall or ring channel 666 and the ridge 668. The first wall or ring channel 664 is configured to receive a first outer wall or ring 670. In one embodiment, the first outer ring 670 may be insertable from a top opening 657 of the guard 656. The second wall or ring channel 666 is located between the first ring channel 664 and the ridge 668. The second ring channel 666 is configured to receive a heating receptacle, such as exemplary heating receptacle 674. In one embodiment, the heating receptacle 674 may be insertable from a top opening 657 of the guard 652. In one embodiment, the ridge 668 forms an aperture 662 sized to receive a heating element, such as exemplary heating element 104, 154, and the ridge 668 may be configured to receive a second wall or ring 674.

Still referring to FIG. 14 and referring back to FIGS. 13A-13D, in some embodiments, the first channel 664, the second ring channel 666 and the ridge 668 are configured to retain a first wall or ring 670, 870, a second wall or ring 672, 872 and a heating receptacle 674, 874. In some embodiments, the second ring channel 666 and the ridge 668 are sized to abut and frictionally hold or retain each of a first wall or ring 670, 870, a second wall or ring 672, 872, and a heating receptacle 674, 874. In some embodiments, an adhesive, such as at least one of a polymer, glue, any chemical bonding agent, and any mechanical bonding agent may be disposed along the channels 664, 666 and the ridge 668 to facilitate retention.

Still referring to FIG. 14 and referring back to FIGS. 13A-13C, the base portion 654 further comprises at least one radial channel 676, the radial channel extending from the outer edge of ridge 668 to the first wall or ring channel 664. In one embodiment, the first wall or ring channel 664 and the second wall or ring channel 666, may be positioned at a different depth relative to the proximal end 660 of the base portion 654. In an embodiment, the first wall or ring channel 664 may be located in the approximately same axial plane as the radial channel 676, where the axial plane is in relation to the shell 652. In one such embodiment, the second wall or ring channel 666 may be located on an elevated axial plane as compared to the radial channel 676. Accordingly, when a first wall or ring 670, 870, a second wall or ring 672, 872, and a heating receptacle 674, 874 are inserted onto or into their respective positions, an air pathway may desirably be formed from the aperture 662, up the inside surface of the second wall or ring 674, to the inside surface of the heating receptacle 674, then down through the at least one radial channel 676 below the heating receptacle 674, and then inside the first wall or ring 670 (FIG. 13A). In some embodiments, the channel 676 may comprise an air-permeable material to prevent physical materials from travelling into the aperture 662 stored in the chamber 650. In a further embodiment, second wall or ring 672 and/or second channel 666 may further comprise one or more radial ribs or connecting spacers (not shown) extending between at least a portion of heating receptacle 674 and second wall or ring 672, which may comprise a suitably heat conductive material, such that heating receptacle 672, 872 may additionally transfer heat to second wall or ring 672, 872 at laest in part by conduction, such as to heat a consumable material held within chamber 650, 850, for example.

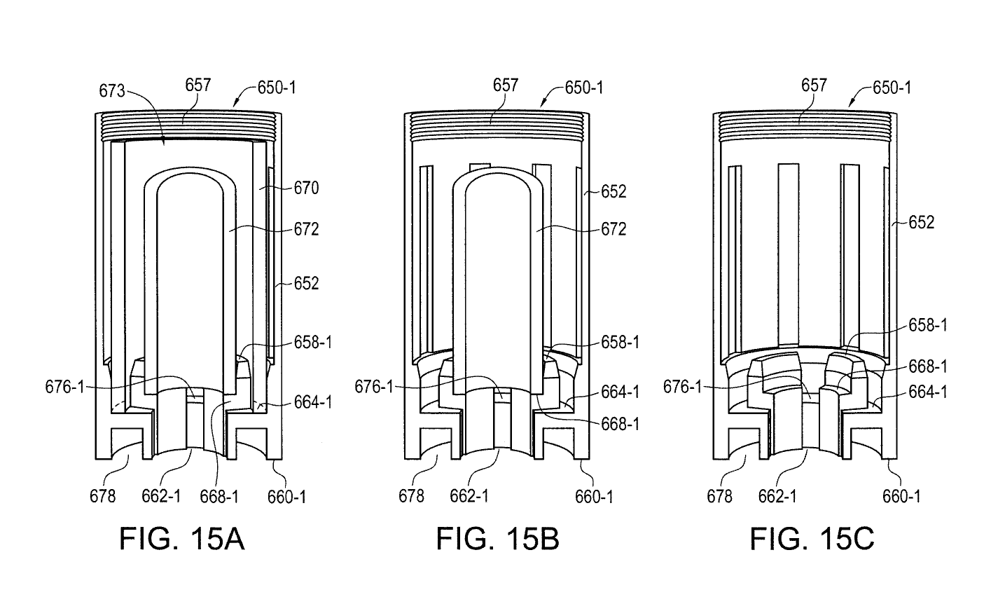

Referring to FIG. 15A, this diagram illustrates, in a cut-away front elevation perspective view, a vaporization chamber 650-1, in accordance with an embodiment of the present disclosure. The chamber 650-1 comprises a protective shell 652, an outer wall or ring 670, and a heating receptacle 674.

Referring to FIG. 15B, this diagram illustrates, in a cut-away front elevation perspective view, the chamber 650-1, as shown in FIG. 15A, with the outer wall or ring 670 removed for furthering understanding of any internal components, in accordance with an embodiment of the present disclosure.

Referring to FIG. 15C, this diagram illustrates, in a cut-away front elevation perspective view, the chamber 650-1, as shown in FIG. 15A, with the outer wall or ring 670 and the middle heating receptacle 674 removed for furthering understanding of any internal components, in accordance with an embodiment of the present disclosure.

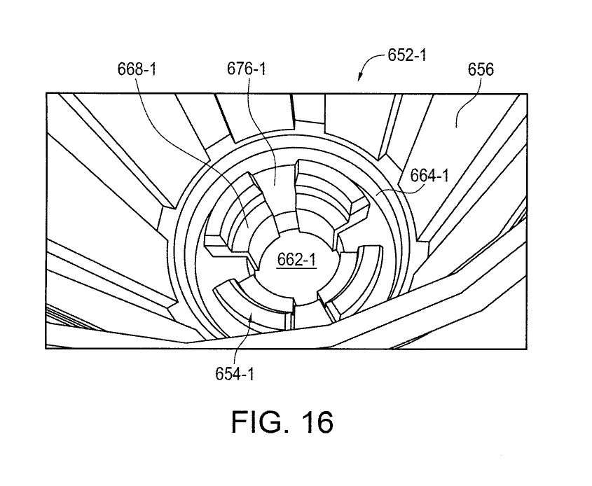

Referring to FIG. 16, this diagram illustrates, in a top perspective view, a protective shell 652-1, having a base portion 654-1 and guard portion 656, in accordance with an embodiment of the disclosure. Still referring to FIG. 16 and referring back to FIGS. 15A-15C, the shell 652-1 comprises a base portion 654-1 and a guard portion 656. The base portion 654-1 has a top surface 658-1 and a bottom surface 660-1. The base portion 654-1 is configured to receive the heating element 104. In one embodiment, the base portion 654-1 comprises a substantially disk shape, having an aperture 662-1 extending from the bottom surface 660-1 through to the top surface 658-1. The aperture 662-1 is sized to receive a heating element, such as exemplary heating element 104, 154. That is, a proximal end of a heating element 104, 154 may be inserted through aperture 662-1. In some embodiments, the base portion 654-1 comprises a ridge 668-1. In this embodiment, the ridge 668-1 defines the outer dimensions of the aperture 662-1, the aperture 662-1 sized to receive a heating element 104, 154.

Still referring to FIG. 16 and referring back to FIGS. 15A-15C, the base portion 654-1 comprises a ring channel 664-1, and a ridge 668-1 on a top surface 658-1. The ring channel 664-1 is configured to receive a first outer wall or ring 670. In one such embodiment, the first outer wall or ring 670 may be insertable from a top opening of guard 656. The ridge 668-1 is configured to retain a heating receptacle 674 that in one embodiment may be insertable from a top opening of the guard 652-1. The base portion 654-1 further comprises at least one radial channel 676-1, the radial channel extending from the aperture 662-1 to the ring channel 664-1. In some embodiments, the ring channel 664-1 and the ridge 668-1 may be positioned at different depths relative to one another and relative to the proximal end 660-1 of the base portion 654-1.

Still referring to FIG. 16 and referring back to FIGS. 15A-15C, in an embodiment, the ring channel 664-1 may be located in approximately the same axial plane as the radial channel 676-1, where the axial plane parallel is in relation to end 660-1. In this embodiment, the ridge 668-1 is located on an elevated axial plane in relation to the radial channel 676-1. Accordingly, when the first wall or ring 670, and the heating receptacle 674, are inserted onto or into their respective positions, an air pathway is formed from the aperture 662-1, through the at least one radial channel 676 below heating receptacle 674, and toward the inside of the first ring 670 (as shown in FIG. 15A). When the first ring 670 and the heating receptacle 674 are inserted into the shell 652, a storage chamber or bore 673 is formed for receiving and storing at least one consumable substance for vaporization. In some embodiments, the channel 676-1 comprises air permeable material to prevent physical materials from travelling into the aperture 662 from the chamber 650. For the remainder of this disclosure, elements denoted with reference numerals having “−1” may be denoted with or without “−1”, those elements referred to interchangeably, unless otherwise expressly stated.

Still referring to FIG. 16 and referring back to FIGS. 13A-13D and 15A-15C, the base portion 654 further comprises a channel 678 configured to receive at least one magnetic object (not shown). In some embodiments, the channel 678 is disposed on a bottom 660 of the base portion 654. In some embodiments, the channel 678 and a magnetic object each comprise a complementary ring-shaped magnetic element, and a magnetic object may be sized for insertion and fixed attachment to the channel 678. In other embodiments, the bottom 660 of the base portion 654 may comprise at least one complementary aperture and a magnet (not shown), the magnet sized for insertion and fixed attachment to the at least one complementary aperture.

Still referring to FIG. 16 and referring back to FIGS. 13A-13C and 15A-15C, in some embodiments, a magnetic object (not shown) may be permanently attached to the channel 678 or the at least one complementary aperture. In these embodiments, an adhesive, such as at least one of a polymer, glue, any chemical bonding agent, and any mechanical bonding agent is applicable between a magnetic object and the channel 678 to the bond the magnetic object together with the base portion 654 of chamber 650. In some embodiments, the base portion 654 may comprise magnetic objects (not shown). In some embodiments, the magnetic objects may be injection-molded into the base portion 654. An inner surface of the ring 670, an outer surface of heating receptacle 674, and a portion of the top surface 658 of the base portion 654 defines a bore 673 extending from a top opening 657 to the base 654 (as shown in FIGS. 13A and 15A). The bore 673 is sized to receive and store consumable substances, e.g., herbal materials, tobacco, vaporizable liquids, cannabis materials, and the like, for vaporization.

Still referring to FIG. 16 and referring back to FIGS. 13A-13D and 15A-15C, the chamber 650 is configured to receive a heating element, such as exemplary heating element 104, 154. Reiterating, the chamber 650 comprises a heating receptacle 672. The heating receptacle 672 extends a length L, toward an opening 657, the length L comprising a range that is approximately equal to or greater than the portion of a heating element 104, 154 extending perpendicularly from top 511 of adapter 510. When the chamber 650 is placed over the heating element 104, 154, the heating receptacle 672 surrounds the top and side portions of the heating element 104, 154. In some embodiments of the present disclosure, the surface of heating receptacle 672 may abut heating element 104. In some embodiments of the present disclosure, the surface of the second ring 674 abuts the heating element 104.

Still referring to FIG. 16 and referring back to FIGS. 13A-13D and 15A-15C, in various embodiments of the present disclosure, when a heating element 104, 154 is operating, heat may be transferred through the heating receptacle 674 and the second wall or ring 672 by at least one of conduction and radiation. The heating receptacle 672 and the second wall or ring 672 transfers the heat to the at least one consumable substance in the bore 673 by at least one of further conduction and further radiation. In some embodiments of the present disclosure, the heating receptacle 674 transfers heat to the base 652 by at least conduction. In some of these embodiments of the present disclosure, the base 652 transfers heat to the first wall or ring 670 by at least conduction. In various embodiments of the present disclosure, heat is transferable towards the at least one consumable substance by at least one of the heating receptacle 674, the base 652, and the first wall or ring 670.

Still referring to FIG. 16 and referring back to FIGS. 13A-13D and 15A-15C, the at least one consumable substance disposed in the bore 673 may desirably be vaporized through at least convection heating. As disclosed elsewhere in this disclosure, air may desirably enter through the aperture 662, travel up the inside surface of the second wall or ring 672, and down the outside surface of the second wall or ring 672 before entering the bore 673. In this embodiment, when the heating element 104, 154 is operating, air is heated while moving along the surface of the second wall or ring 672. Accordingly, the heated air, upon entering the bore 673, causes convective heating of the at least one consumable substance disposed within the bore 673.

Still referring to FIG. 16 and referring back to FIGS. 13A-13D and 15A-15C, in one embodiment, the shell 652 may comprise a reflective material for reflecting heat that is emitted from the heating element 104 back toward the at least one consumable substance stored within the bore 673. In some embodiments, the shell 652 may comprise an internal portion for reflecting heat and an external portion comprising a desirably low thermal conductivity material for protecting a user from the heat emitted by the heating element 104, 154. In some embodiments, the low thermal conductivity material comprises a thermal conductivity in a range of less than approximately 1 W/mK. In some embodiments, the low thermal conductivity material comprises a thermal conductivity in a range of less than approximately 10 W/mK.

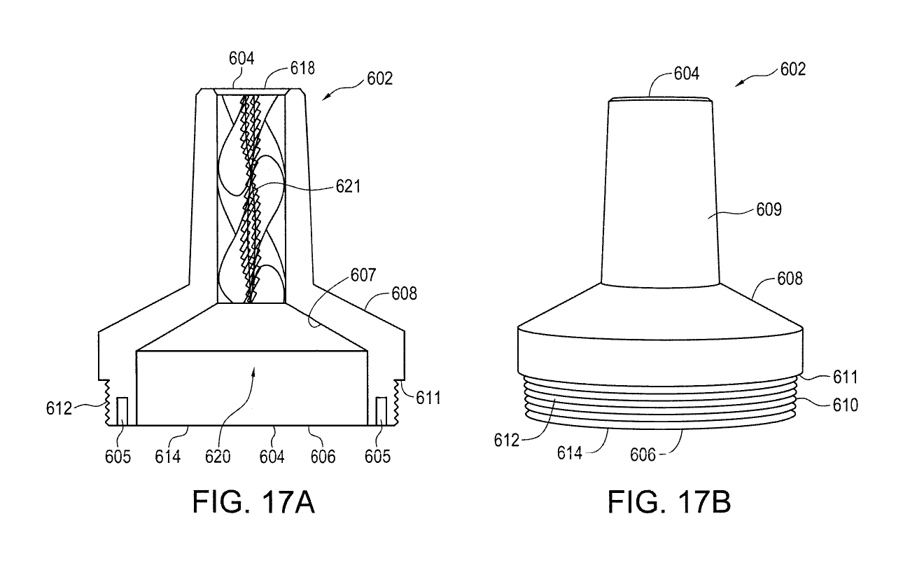

Referring to FIG. 17A, this diagram illustrates, in a cut-away front perspective view, a mouthpiece 602, in accordance with an embodiment of the present disclosure. The mouth piece 602 has a top 604, a bottom 606, an inner surface 607, and an outer surface 608. The inner surface 607 comprises a bore 620 having a substantially conical, or a funnel, shape along a vertical axis. The inner surface 607 is further defined by the surface of a vapor-permeable base 614 facing top 604. The bore 620 has a narrow top opening 618 located at the top 604. The bore 620 comprises a substantially circular shape, by example only. The bore 620 may, generally, be any 3-dimensional geometric space having a vapor-permeable base and an opening. In some embodiments, the bore 620 comprises at least one air path winding throughout bore 620 that increases the travelling distance for air or vapor to enter the bore 620 and the exit top opening 618. The increased travel distance allows additional time for the vapor to cool as the vapor enters the bore 620 and exits the opening 618. In some embodiments, a plurality of air paths 621 intertwine in a double helix formation, thereby increasing the travelling distance for air to travel within mouthpiece 602.

Referring to FIG. 17B, this diagram illustrates, in front perspective view the mouthpiece 602, as shown in FIG. 17A, in accordance with an embodiment of the present disclosure. The top opening 618 is circularly shape and sized to comfortably insert into a user's mouth. The top opening 618 comprises a circular shape by example only. The top opening 618 comprises any two-dimensional closed shape, such as a circle, half circle, polygon, moon-shaped, and the like. In some embodiments of the present disclosure, the surface area of opening 618 comprises a range from approximately 0.75 cm² to approximately 20 cm². In other embodiments of the present disclosure, the surface area of the opening 618 comprises a range from approximately 1.75 cm² to approximately 7 cm². In some embodiments of the present disclosure, the opening 618 is adapted to couple with at least one of mouth pieces, hoses, tubes, whips, conduits, adapters, and the like.

Still referring to FIGS. 17A and FIG. 17B, in some embodiments of the present disclosure, the mouthpiece 602 comprises at least one of glass, ceramic, plastic, metal, and other suitable material. In some embodiments, the mouthpiece 602 comprises a recyclable material. In an embodiment of the present disclosure, the outer surface 608 of the mouthpiece 602 comprises an upper surface 609 and a recessed surface 610. The upper surface 609 is the portion of the outer surface 608 extending from opening 618 to ridge 611. A recessed surface is the portion of the outer surface 608 extending from the ridge 611 to the edge of the base 614.

Still referring to FIGS. 17A and FIG. 17B and referring back to FIGS. 12A and 12B, a separator or vapor-permeable base 614 prevents herbs and other consumable substances from entering the bore 620 of mouthpiece 620 (as shown in FIG. 12A). In some embodiments of the present disclosure, the separator or vapor-permeable base 614 comprises a material distinct from the remaining portions of mouthpiece 602. In these embodiments of the present disclosure, the separator or vapor-permeable base 614 comprises at least one of a mesh material, an interlaced material, and a vapor-permeable fabric, such cotton or polyester. The separator is sized to at least cover the internal bore 673 of first ring 670. In some embodiments, the mouthpiece 602 comprises a separator or vapor-permeable base 614. In some embodiments, the mouthpiece 602 further comprises a separator receptacle 605 configured to receive a separator or vapor-permeable base 614. In this embodiment, a portion of the proximal outer ends of the separator or vapor-permeable base 614 are insertable into the separator receptacle 605.

Still referring to FIGS. 17A and FIG. 17B and referring back to FIGS. 12A and 12B, the mouthpiece 602 is configured to couple with the chamber 650. In an embodiment of the present disclosure, the outer dimension of the recessed surface 610 comprises a range that is approximately equal to, or less than, the inner dimension of the guard 652, wherein the mouthpiece 602 is insertable into the guard 652 of the chamber 650. When the bottom end 606 of the mouthpiece 602 is inserted into the guard 652 of the chamber 650, the ridge 611 of the mouthpiece 602 abuts the top edge of the guard 652.

Still referring to FIG. 17A and FIG. 17B and referring back to FIGS. 12A and 12B, in some embodiments of the present disclosure, the mouthpiece 602 is removably attachable to the chamber 650. In some embodiments of the present disclosure, when mouthpiece 602 is attached to the chamber 650, a seal is formed between the mouthpiece 602 and the chamber 650. In an embodiment of the present disclosure, the recessed surface 610 comprises at least one seal receptacle 612 sized to receive at least one seal, such as an O-ring. The at least one seal is disposable relative to the at least one seal receptacle 612. When the mouthpiece 602 is inserted into the chamber 650, the recessed surface 610 is adjacent an inner surface of the guard 652; and a seal is positioned between the adjacent surfaces of the mouthpiece 602 and the chamber 650, thereby forming a vapor seal, and further frictionally engaging the mouth piece 602 with the chamber 650. The mouthpiece 602 is removable from the chamber 650 by exerting a sufficient axial force. In other embodiments of the present disclosure, other various pressure-fit or interference-fit techniques may be employed to removably attach the mouthpiece 602 to the chamber 650. In some embodiments, the recessed surface 610 and the top portion 657 of the guard 652 comprise complementary threads for attaching the mouthpiece 602 to the chamber 650.

Still referring to FIG. 17A and FIG. 17B and referring back to FIGS. 12A and 12B, in an embodiment of the present disclosure, a cartridge 600 is provided, the cartridge 600 comprising a disposable cartridge, wherein cartridge 600 further comprises a mouthpiece 602, a chamber 650, and at least one consumable substance, wherein the at least one consumable substance is predisposed in the bore 673 of the chamber 650. A separator or vapor-permeable base 614 is placed between the bore 673 and the bore 620, thereby preventing the at least one consumable substance from entering bore 620. In some embodiments, the mouthpiece 602 is then permanently sealably attached to the chamber 650 via at least one seal, such as an O-ring, a tamper seal, an adhesive, such as at least one of a polymer, glue, any chemical bonding agent, and any mechanical bonding agent. In some embodiments, mouthpiece 602 is removably attached to the chamber 650 via complementary threaded portions.

Still referring to FIG. 17A and FIG. 17B and referring back to FIGS. 12A and 12B, in some embodiments of the present disclosure, the disposable cartridge 600 further comprises vapor seals. In these embodiments, vapor seals may be place over the top 618 of the mouthpiece 602 and the aperture 662 of the chamber 650. The vapor seals maintain the freshness of the at least one consumable substances that is pre-disposed in the bore 673 of the chamber 650 for an extended time by preventing oxidation. In some embodiments, the disposable cartridge 600 further comprises inert gases for delaying oxidation of the at least one consumable substance stored within the chamber 650.

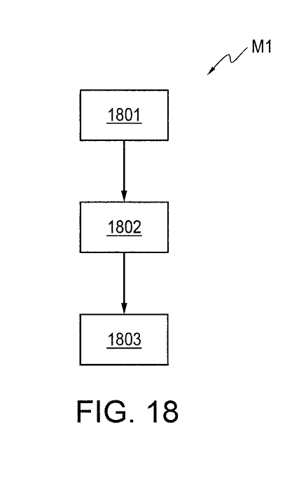

Referring to FIG. 18, this flowchart illustrates a method M1 of fabricating a portable non-combustion vaporizer device 2, 4 for generating vapors, the method M1 comprising: providing a heating component 100, as indicated by block 1801; and providing a cartridge 300, as indicated by block 1802, in accordance with an embodiment of the present disclosure. The heating component providing 1801 comprises providing the heating element 100 as substantially perpendicularly extending therefrom and into the cartridge 300. In an embodiment of the present disclosure, the cartridge providing 1802 comprises providing the cartridge 300 as removably attachable in relation to the heating component 100. In an embodiment of the present disclosure, the method M1 of fabricating the portable non-combustion vaporizer device 2, 4 further comprises providing a collar 200 for facilitating removably attaching the cartridge 300 to the heating component 100, as indicated by block 1803.

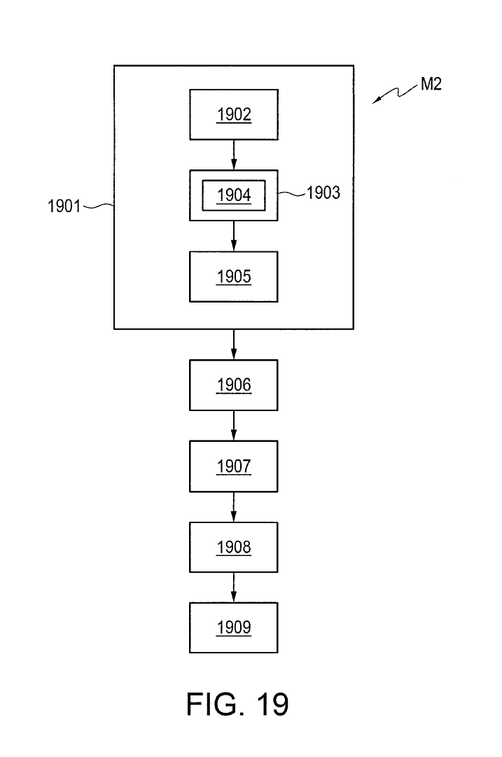

Referring to FIG. 19, this flowchart illustrates a method M2 of generating vapors by way of a portable non-combustion vaporizer device 2, 4, the method M2 comprising providing the vaporizer device 2, 4, 9 as indicated by block 1901, the device providing 1901 comprising: providing a heating component 100, 150, as indicated by block 1902; and providing a cartridge 300, 700, as indicated by block 1903, in accordance with an embodiment of the present disclosure. The heating component 100, 150 providing 1901 comprises providing the heating element 100, 150 as substantially perpendicularly extending therefrom and into the cartridge 300, 700. In an embodiment of the present disclosure, the cartridge providing 1903 comprises providing the cartridge 300, 700 as removably attachable in relation to the heating component 100, 150. The cartridge 300, 700 providing 1903 comprises loading the cartridge 300, 700, as indicated by block 1904, wherein loading 1904 the cartridge 300, 700 comprises loading at least one consumable substance (not shown).

Still referring to FIG. 19, in an embodiment of the present disclosure, providing 1901 the portable non-combustion vaporizer device 2, 4 further comprises providing a collar 200 for facilitating removably attaching the cartridge 300 to the heating component 100, as indicated by block 1905. The method M2 of generating vapors further comprises coupling the cartridge 300 with the heating component 100 by way of the collar 200, as indicated by block 1906. The method M2 of generating vapors further comprises providing a mouthpiece 302, as indicated by block 1907; and coupling the mouthpiece 302 with the collar 200, as indicated by block 1908. The method M2 of generating vapors further comprises powering the heating component 100, thereby applying heat to the at least one consumable substance disposed in the cartridge 300, and thereby vaporizing the at least one consumable substance, as indicated by block 1909.

Information as herein shown and described in detail is fully capable of attaining the above-described object of the present disclosure, the presently preferred embodiment of the present disclosure, and is, thus, representative of the subject matter which is broadly contemplated by the present disclosure. The scope of the present disclosure fully encompasses other embodiments which may become obvious to those skilled in the art, and is to be limited accordingly, by nothing other than the appended claims, wherein any reference to an element being made in the singular is not intended to mean “one and only one” unless explicitly so stated, but rather “one or more.” All structural and functional equivalents to the elements of the above-described preferred embodiment and additional embodiments as regarded by those of ordinary skill in the art are hereby expressly incorporated by reference and are intended to be encompassed by the present claims.

Moreover, no requirement exists for a system or method to address each and every problem sought to be resolved by the present disclosure, for such to be encompassed by the present claims. Furthermore, no element, component, or method step in the present disclosure is intended to be dedicated to the public regardless of whether the element, component, or method step is explicitly recited in the claims. However, that various changes and modifications in form, material, work-piece, and fabrication material detail may be made, without departing from the spirit and scope of the present disclosure, as set forth in the appended claims, as may be apparent to those of ordinary skill in the art, are also encompassed by the present disclosure.

The exemplary embodiments herein described are not intended to be exhaustive or to limit the scope of the disclosure to the precise forms disclosed. They are chosen and described to explain the principles of the disclosure and its application and practical use to allow others skilled in the art to comprehend its teachings.

As will be apparent to those skilled in the art in light of the foregoing disclosure, many alterations and modifications are possible in the practice of this disclosure without departing from the scope thereof.

What is claimed is:

1. A vaporizer cartridge comprising a base, inner and outer substantially concentric walls extending from said base and defining a vaporization chamber between said outer and inner walls adapted for containing at least one vaporizable substance, and a heating receptacle opening extending through said base within a perimeter of said inner wall adapted for receiving a heating element inserted into said heating receptacle opening, wherein a top edge of said outer wall forms an open top rim adapted for coupling to a mouthpiece.

2. The vaporizer cartridge according to claim 1 additionally comprising a mouthpiece comprising a top opening, a central open bore, and a bottom opening sealably coupled to said open top rim of said cartridge.

3. The vaporizer cartridge according to claim 2 additionally comprising at least one seal situated between said bottom opening of said mouthpiece and said open top rim of said cartridge.

4. The vaporizer cartridge according to claim 2 wherein said mouthpiece is releasably coupled to said open top rim of said cartridge.

5. The vaporizer cartridge according to claim 2 wherein said mouthpiece is lockably coupled to said open top rim of said cartridge.

6. The vaporizer cartridge according to claim 5 wherein said mouthpiece additionally comprises one or more mechanical locking members adapted to lockingly couple said mouthpiece to said open top rim of said cartridge.

7. The vaporizer cartridge according to claim 2 additionally comprising at least one seal adapted to removably seal said top opening of said mouthpiece.

8. The vaporizer cartridge according to claim 1 wherein said base additionally comprises at least one air aperture adapted to admit air into said vaporization chamber.

9. The vaporizer cartridge according to claim 1 additionally comprising a guard layer surrounding at least a portion of said outer wall, wherein said guard layer is adapted to at least partially thermally insulate a user grasping said guard layer from said outer wall.

10. The vaporizer cartridge according to claim 1, additionally comprising a heating receptacle wall extending from said heating receptacle opening and disposed substantially concentrically within said inner wall, wherein said heating receptacle wall is adapted for receiving a heating element inserted into said heating receptacle through said heating receptacle opening in said base.

11. The vaporizer cartridge according to claim 10, wherein at least one of said outer wall, said inner wall and said heating receptacle wall comprise at least one thermally conductive material selected from the list comprising: glass, metal, polymer, ceramic and composite materials.

12. The vaporizer cartridge according to claim 1, additionally comprising at least one electronically readable memory device attached to said cartridge.

13. The vaporizer cartridge according to claim 12, wherein said at least one electronically readable memory device comprises at least one of: a printed addressable memory device, a thin film printed memory device, a radio frequency identification device, a barcode and a magnetic memory device.

14. The vaporizer cartridge according to claim 1 additionally comprising at least one vaporizable material contained in said vaporization chamber.

15. The vaporizer cartridge according to claim 14, wherein said at least one vaporizable substance comprises a consumable substance comprising at least one of: herbal, tobacco, cannabis, and glycol-based liquid materials.

16. A vaporizer apparatus comprising a vaporizer cartridge according to claim 1, and a heating component comprising a heating element extending from a top surface thereof and circuitry adapted to control heating of said heating element, wherein said heating element is inserted into the heating receptacle opening in the base of said vaporizer cartridge and said vaporizer cartridge is coupled to said top surface of said heating component.

17. The vaporizer apparatus according to claim 16, wherein said vaporizer cartridge additionally comprises a mouthpiece comprising a top opening, a central open bore, and a bottom opening sealably coupled to said open top rim of said cartridge.

18. The vaporizer apparatus according to claim 16, wherein said circuitry comprises a battery and a power switch adapted for activation by a user.

19. The vaporizer apparatus according to claim 16, wherein said heating component is adapted for handheld use by a user.

20. The vaporizer apparatus according to claim 16, wherein said vaporizer cartridge additionally comprises at least one electronically readable memory device attached to said cartridge, and wherein said heating component additionally comprises at least one electronic memory device reader adapted to interface with said electronically readable memory device attached to said cartridge.

21. The vaporizer apparatus according to claim 16, wherein said vaporizer cartridge is removably coupled to said heating component and is adapted for manual removal and replacement by a user.

22. A method of making a vaporizer cartridge, comprising:
providing a base, inner and outer substantially concentric walls extending from said base and defining a vaporization chamber between said outer and inner walls adapted for containing at least one vaporizable substance, and a heating receptacle opening extending through said base within a perimeter of said inner wall adapted for receiving a heating element inserted into said heating receptacle opening, wherein a top edge of said outer wall forms an open top rim adapted for coupling to a mouthpiece
filling at least a portion of said vaporization chamber with at least one vaporizable substance; and
sealably coupling a mouthpiece comprising a top opening, a central open bore, and a bottom opening to said open top rim of said cartridge.

23. The method of making a vaporizer cartridge according to claim 23, additionally comprising attaching at least one electronically readable memory device to the vaporizer cartridge.

24. The method of making a vaporizer cartridge according to claim 23, additionally comprising lockingly coupling said mouthpiece to said open top rim of said cartridge.

25. A method of vaporizing a consumable substance, comprising:
providing a vaporizer cartridge comprising a base, inner and outer substantially concentric walls extending from said base and defining a vaporization chamber between said outer and inner walls and containing at least one vaporizable consumable substance, and a heating receptacle opening extending through said base within a perimeter of said inner wall adapted for receiving a heating element inserted into said heating receptacle opening, wherein a top edge of said outer wall forms an open top rim sealably coupled to a mouthpiece;
providing a heating component comprising a heating element extending from a top surface thereof and circuitry adapted to control heating of said heating element;
coupling said vaporizer cartridge to said heating element such that said heating element is inserted into the heating receptacle opening in the base of said vaporizer cartridge; and
activating said heating component to heat said heating element and vaporize said at least one consumable substance.
